(19) United States
(12) Patent Application Publication
(10) Pub. No.: US 2021/0244645 A1
(43) Pub. Date: Aug. 12, 2021

(54) ORAL CARE COMPOSITION AND DEVICES FORMED THEREWITH

(71) Applicant: Ranir, LLC, Grand Rapids, MI (US)

(72) Inventors: Kristy CAREY, Grand Rapids, MI (US); Alan THELEN, Bushnell, FL (US); Michael WRIGHT, Ada, MI (US)

(21) Appl. No.: 16/788,568

(22) Filed: Feb. 12, 2020

(51) Int. Cl. A61K 8/81; A61K 8/86; A61K 8/22; A61K 8/42; A61K 8/49; A61C 19/06; A61Q 11/00

(52) U.S. Cl. CPC A61K 8/8176; A61K 8/8147; A61K 8/86; A61K 8/22; A61K 8/42; A61K 2800/92; A61C 19/066; A61Q 11/00; A61K 2800/48; A61K 2800/524; A61K 8/492

(57) ABSTRACT

An oral care composition is disclosed. The oral care composition includes (A) an adhesive agent comprising an alkylated polyvinylpyrrolidone, (B) a miscibility component comprising an acrylic acid-acrylate copolymer, and (C) a plasticizer comprising a polyalkylene glycol. An oral care device is also disclosed. The oral care device includes an oral care layer comprising the oral care composition and a whitening agent. A method of preparing the oral care device is also disclosed, and includes extruding the oral care composition to give the oral care layer, and preparing a laminate comprising the oral care layer. The method may further include processing the laminate to give the oral care device, which may be prepared as an oral care strip or an oral care tray.

### FIELD OF THE INVENTION

The present disclosure relates generally to oral care compositions and, more specifically, to oral care compositions for preparing oral care devices for whitening teeth and methods of preparing and utilizing the same.

### DESCRIPTION OF THE RELATED ART

Oral care devices are employed to deliver cosmetic and/or therapeutic agents (i.e., active agents) to various surfaces in the oral cavity. For example, some such devices (e.g. dental devices) are used to deliver active agents to teeth, which comprise a hard and slightly-porous outer enamel layer disposed about a softer inner dentin layer. In particular, the enamel layer is susceptible to impregnation and/or pigmentation from various compounds (e.g. tannins, polyphenols, etc.), which are present in many common consumables, often leading to tooth discoloration of teeth. While such tooth discoloration is widely occurring, it is often stigmatized and thus can negatively affect a person's wellbeing. Accordingly, many active agents have been discovered and/or developed to ameliorate tooth discoloration and are frequently delivered/administered to a person's teeth via a dental device, which are also known as tooth-whitening devices/appliances. Examples of such dental devices (i.e., tooth-whitening devices) generally include strips and trays, which are disposed about teeth and used to deliver an oral care agent (e.g. a bleaching or whitening agent) thereto. Such strips and trays may be single or multi-layered, and often include an adhesive to facilitate positioning the devices about the teeth during application.

Unfortunately, many conventional dental devices suffer from numerous drawbacks associated with their design and/or material composition. For example, such devices require an outer layer (i.e., a layer to contact teeth) that comprises a mobile active agent, such as a bleaching or whitening agent, which may irritate or damage tissues on a person's hands, gums, etc. To minimize contact with such tissues, many devices are one-sided (i.e., include a tooth contact side). However, some such devices suffer from misapplication, e.g. “wrong-side” application of the strip to the teeth, which may result in contact of the tooth contact side, and thus the active agent, to other tissues. Other devices, such as trays designed to cup a person's teeth, typically utilize an active composition that must be disposed in the tray prior to disposing the tray about a person's teeth. Appropriate application of the active composition can minimize incidental exposure of peripheral tissues in the oral cavity to the active agent being utilized. However, these trays are typically limited to use in the professional teeth bleaching market, e.g. by dentists and other professionals, as improper dosage of the active composition and/or positioning of the tray (e.g. by an inexperienced “at-home” user) results in less efficacious treatment, incidental exposure and/or ingestion of the whitening composition, etc.

Recently, some at-home (i.e., over-the-counter) dental devices have been developed to include a pre-measured amount of an active agent that is delivered to a person's tooth, without need for exposure of tissues to a contact tooth contact side. These devices are generally configured as a composite multi-layered flexible strip, which includes an adhesive layer and a backing layer. The adhesive layer typically includes the active agent to be delivered, and the backing layer is generally formulated to be impermeable to the active agent and other components of the adhesive layer. More recently, drawbacks associated with the end-use of such designs, including delamination during use, premature inactivation of the active agent, inability for manipulation/realignment during use from premature deactivation of the adhesive, etc., have been overcome with success. However, these devices still suffer from many production-side disadvantages, including costly and difficult steps in formulating active compositions, formation of the composite strips, and storage of light and/or moisture-sensitive components.

### BRIEF SUMMARY OF THE INVENTION

An oral care composition is provided. The oral care composition includes (A) an adhesive agent comprising an alkylated polyvinylpyrrolidone, (B) a miscibility component comprising an acrylic acid-acrylate copolymer, and (C) a plasticizer comprising a polyalkylene glycol.

An oral care device is also provided. The oral care device includes an oral care layer comprising the oral care composition and a whitening agent.

A method of preparing the oral care device is further provided. The method includes extruding the oral care composition to create the oral care layer, and laminating the oral care layer with a substrate and a release liner such that the oral care layer is disposed between the substrate and the release liner, thereby preparing the oral care device. The oral care device may be prepared as an oral care strip or an oral care tray using a rollstock laminate provided by the method.

These and other features and advantages of the present disclosure will become apparent from the following description of particular embodiments, when viewed in accordance with the accompanying drawings and appended claims.

### BRIEF DESCRIPTION OF THE DRAWINGS

FIG. 1 is planar view of an oral care strip;

FIG. 2 is an isometric view of an oral care tray;

FIG. 3 is a cross-sectional view of an oral care laminate; and

FIG. 4 is a cross-sectional view of another oral care laminate.

### DETAILED DESCRIPTION OF THE INVENTION

The present disclosure provides an improved oral care composition, an improved oral care device comprising the composition, and methods of making and using the same. As will be understood in view of the description herein, the disclosed composition(s), article(s), and related method(s) are suitable for use in various end-use applications involving cosmetics and/or personal care and, specifically, those involving oral care, dental treatments, and the like. The formulation of the oral care composition provides increased processability and utility (e.g. via decreased processing temperatures, improved flow (e.g. melt-flow) properties, improved film-forming properties, increased flexibility, cohesion, and adhesiveness, and decreased brittleness in film), such that the disclosed oral care composition, the oral care device formed therewith, and the methods involving the same, provide numerous advantageous and improvements, as illustrated by the description herein.

As introduced above, an oral care composition (the “composition”) is provided. In general, the composition comprises (A) an adhesive agent, (B) a miscibility component, and (C) a plasticizer, which are each described in detail below. However, the composition may comprise additional components, such as inactive, active, and/or adjuvant components, or may be combined with other components or formulations, depending on an intended use of the composition, as will be appreciated from the description below and as understood by one of skill in the art.

The adhesive agent (A) comprises an alkylated polyvinylpyrrolidone. In general, the alkylated polyvinylpyrrolidone of the adhesive agent (A) comprises a polyvinylpyrrolidone (PVP) polymer (i.e., a compound having a backbone including subunits derived from a vinylpyrrolidone monomer, such as N-vinyl-2-pyrrolidone) having at least one alkyl group appended thereto. Typically, the polyvinylpyrrolidone polymer comprises subunits derived from an N-vinylpyrrolidone monomer, such as those having an ethenyl-2-pyrrolidone skeleton. In certain embodiments, the polyvinylpyrrolidone polymer is a homopolymer of N-vinylpyrrolidone (i.e., consists of ethenyl-2-pyrrolidone-based subunits). The polyvinylpyrrolidone polymer may comprise the alkyl group, which is described in further detail below, as a terminal or pendant group (i.e., bonded to the backbone of the polymer), as a functional group bonded to a pendant group (e.g. a pyrrolidone group), or both. As such, the alkyl group may be appended to the polyvinylpyrrolidone polymer after its formation (e.g. via post-polymerization alkylation), prior to formation (i.e., via pre-polymerization alkylation of one or more monomers, which are then polymerized to prepare the polyvinylpyrrolidone polymer and/or the alkylated polyvinylpyrrolidone), or both. In certain embodiments, the alkylated polyvinylpyrrolidone comprises a polyvinylpyrrolidone polymer that is alkylated concurrently with polymerization of N-vinylpyrrolidone monomer(s) (i.e., is a graft copolymer of a polyvinylpyrrolidone and an alkylating compound, such as an alkene). Examples of methods of preparing such compounds can be found in U.S. Pat. No. 3,423,381, which is incorporated by reference herein.

In certain embodiments, the alkylated polyvinylpyrrolidone comprises a moiety (e.g. a subunit and/or segment) having the following general formula (I):

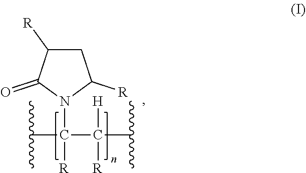

where each R is independently H or an alkyl group, with the proviso that at least one R is the alkyl group, and subscript n 2.

In general, each R is independently selected in each moiety indicated by subscript n. As introduced above, at least one R is the alkyl group. Typically, the alkylated polyvinylpyrrolidone comprises an average of at least one, alternatively at least two, of the alkyl group in each moiety indicated by subscript n (i.e., a degree of substitution (DS) of at least 1, alternatively at least 2, where the DS is the average number of alkyl groups attached per moiety indicated by subscript n). However, as will be understood in view of general formula (I) above, the DS may be any positive value up to 4. Said differently, the alkylated polyvinylpyrrolidone may comprise a DS of from greater than 0 to 4, such as from 0.1 to 4, alternatively from 0.5 to 4, alternatively from 0.5 to 3.5, alternatively of from 0.5 to 3, based on the number of ethenyl-2-pyrrolidone-based subunits indicated by subscript n.

Examples of alkyl groups suitable for R include methyl, ethyl, propyl (e.g. iso-propyl and/or n-propyl), butyl (e.g. isobutyl, n-butyl, tert-butyl, and/or sec-butyl), pentyl (e.g. isopentyl, neopentyl, and/or tert-pentyl), hexyl, as well as branched saturated hydrocarbon groups (e.g. those having from 6 to 18 carbon atoms). In particular embodiments, the alkyl group is a butyl group, such that the alkylated polyvinylpyrrolidone comprises, alternatively is, a butylated polyvinylpyrrolidone. Likewise, in some such embodiments, each R is independently H or a butyl group.

Subscript n is at least 2, and is otherwise not particularly limited. For example, subscript n may be from 2 to 1,000, such as from 10 to 800, alternatively from 25 to 600, alternatively from 50 to 400.

It is to be appreciated that the alkylated polyvinylpyrrolidone may comprise other subunits and/or segments in additional to those derived from the N-vinylpyrrolidone (e.g. those of general formula I above). Examples of such other subunits include those derived from vinyl monomers, such as N-vinyl lactams (i.e., other than vinylpyrrolidone), vinyl esters (e.g. acrylate, methacrylate), vinyl carboxylates or acids (e.g. acrylic acid, methacrylic acid), vinyl amides, vinyl alcohols, etc., and other olefins such as terminal alkenes (e.g. ethylene, 1-propylene, 1-butylene, etc.), internal alkenes, etc. These subunits may be repeating or non-repeating. As such, the alkylated polyvinylpyrrolidone may comprise segments comprising, alternatively consisting essentially of, alternatively consisting of, a poly(N-vinyl lactam) (i.e., other than poly(N-vinyl-2-pyrrolidone)), a poly(N-vinyl acrylamide), a poly(N-alkylarylamide), a polyacrylate or poly methacrylate, a polyacrylic acid or polymethacrylic acid, a polyvinyl alcohol, a polyvinylamine, a polyalkylene, and the like, as well as copolymers, derivatives, modifications, and combinations thereof. In certain embodiments, the alkylated polyvinylpyrrolidone comprises, alternatively consists essentially of, subunits derived from N-vinylpyrrolidone and an alpha-olefin, such as 1-butene.

Typically, the alkylated polyvinylpyrrolidone comprises a molecular weight of 10,000 to 60,000 Daltons, such as from 15,000 to 50,000, alternatively of from 30,000 to 50,000, alternatively of from 35,000 to 45,000, Daltons.

In certain embodiments, the adhesive agent (A) comprises more than one alkylated polyvinylpyrrolidone, such as 2, 3, 4, or more alkylated polyvinylpyrrolidone. In such embodiments, each alkylated polyvinylpyrrolidone is independently selected, and may be the same as or different from any other alkylated polyvinylpyrrolidone.

The alkylated polyvinylpyrrolidone may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent (e.g. aromatic hydrocarbons such as benzene, toluene, xylene, etc.; aliphatic hydrocarbons such as heptane, hexane, octane, etc.; halogenated hydrocarbons such as dichloromethane, 1,1,1-trichloroethane, chloroform; etc.; ethers such as diethyl ether, tetrahydrofuran, etc.), a silicone fluid, or the like, or combinations thereof. When utilized, the carrier vehicle will be selected based on the particular components of the oral care composition and/or the adhesive agent (A), such as the particular alkylated polyvinylpyrrolidone selected. It will be appreciated that the alkylated polyvinylpyrrolidone may be combined with the carrier vehicle, if utilized, prior to, during, or after being combined with any other components of the adhesive agent (A). In certain embodiments, the adhesive agent (A) is free from, alternatively substantially free from carrier vehicles.

The adhesive agent (A) may be present in the composition in any amount, which will be selected by one of skill in the art, e.g. dependent upon the particular components (A), (B), and (C) selected, etc. Typically, the amount of adhesive agent (A) is selected based on the concentration of the alkylated polyvinylpyrrolidone therein. More specifically, the composition typically comprises an amount of the adhesive agent (A) to comprise the alkylated polyvinylpyrrolidone in an amount of from 30 to 60 wt. %, based on the total weight of the composition. In specific embodiments, the composition comprises the adhesive agent (A), alternatively the alkylated polyvinylpyrrolidone thereof, in an amount of from 40 to 60 wt. %, such as from 40 to 50, alternatively from 40 to 45 wt. %, based on the total weight of the composition.

The miscibility component (B) comprises an acrylic acid-acrylate copolymer. In general, the acrylic acid-acrylate copolymer of the miscibility component (B) is a polymer formed from an acrylate ester monomer and an acrylic acid monomer. Examples of suitable acrylate ester monomers generally include methyl (meth)acrylate, ethyl (meth)acrylate, n-propyl (meth)acrylate, iso-propyl (meth)acrylate, n-butyl (meth)acrylate, iso-butyl (meth)acrylate, sec-butyl (meth)acrylate, and tert-butyl (meth)acrylate, methyl acrylate, ethyl acrylate, n-propyl acrylate, iso-propyl acrylate, n-butyl acrylate, iso-butyl acrylate, sec-butyl acrylate, and tert-butyl acrylate. In certain embodiments, the acrylate ester monomer is selected from methyl acrylate, ethyl acrylate, methyl (meth)acrylate, and ethyl (meth)acrylate. In particular embodiments, the acrylate ester monomer is ethyl acrylate. General examples of the acrylic acid monomer include carboxylic acid forms of the esters above, e.g. acrylic acid, methacrylic acid, etc. In certain embodiments, the acrylic acid monomer is selected from acrylic acid and methacrylic acid. In particular embodiments, the acrylate ester monomer is methacrylic acid. As will be appreciated by those of skill in the art, such acrylic acid-acrylate copolymers comprise a pH-dependent solubility, which may be selected and/or tuned by controlling the relative proportions of ester monomers to acid monomers.

In certain embodiments, the acrylic acid-acrylate copolymer comprises a moiety having the following general formula (II):

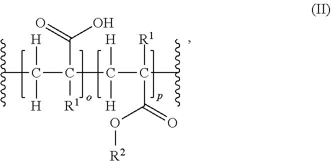

where each R¹ is independently H or an alkyl group, each R² is an independently selected alkyl group, subscript o is ≥2, and subscript p is ≥2. Examples of alkyl groups suitable for R¹ and R² include any of those described herein. For example, in particular embodiments, each R¹ is independently H or a methyl group and each R² is independently C1-C3 hydrocarbyl group such as a methyl, ethyl, or propyl group. In specific embodiments, R¹ is CH₃ in each moiety indicated by subscript o, R¹ is H in each moiety indicated by subscript p, and each R² is CH₂CH₃.

Subscripts o and p are each at least 2, and are otherwise not particularly limited. As will be appreciated by those of skill in the art, subscripts o and p represent the number of acrylic acid monomers and acrylate monomers, respectively, in the acrylic acid-acrylate copolymer (i.e., such that o+m may be the degree of polymerization (DP) of the acrylic acid-acrylate copolymer). It will also be appreciated that the moieties indicated by subscripts o and p in general formula (II) may be in any order (e.g. in randomized and/or block from, etc.), such that the acrylic acid-acrylate copolymer may be a random copolymer, a block copolymer, etc. In general, subscripts o and p are selected in view of one another such that the acrylic acid-acrylate copolymer comprises a ratio of free carboxyl groups to ester groups (i.e., an acid:ester ratio) of from 1:0.5 to 1:3, such as from 1:1 to 1:2. In specific embodiments, the acid:ester ratio of the acrylic acid-acrylate copolymer is from 1:0.9 to 1:1.1, alternatively is 1:1. As such, the acrylic acid-acrylate copolymer may comprise any DP, such as a DP of from 4 to 5,000, alternatively from 100 to 4000, alternatively from 200 to 3000, alternatively from 300 to 2,000. For example, subscript o may be from 2 to 1,000, such as from 10 to 800, alternatively from 25 to 800, alternatively from 50 to 600. Likewise, subscript p may be from 2 to 1,000, such as from 10 to 800, alternatively from 25 to 800, alternatively from 50 to 600.

It is to be appreciated that the acrylic acid-acrylate copolymer may comprise other subunits and/or segments in additional to those derived from the acrylate esters and acrylic acids. Examples of such other subunits include those derived from vinyl monomers, such as any of those described above, which may be repeating or non-repeating.

Typically, the acrylic acid-acrylate copolymer comprises a molecular weight (e.g. weight average, as a determined by gel permeation chromatography) of from 80,000 to 200,000 Daltons, such as from 90,000 to 180,000, alternatively of from 100,000 to 170,000, alternatively of from 110,000 to 150,000, alternatively of from 120,000 to 150,000 Daltons. In specific embodiments, the acrylic acid-acrylate copolymer comprises a molecular weight of from 130,000 to 140,000, alternatively of 135,000, Daltons.

In certain embodiments, the miscibility component (B) comprises more than one acrylic acid-acrylate copolymer, such as 2, 3, 4, or more acrylic acid-acrylate copolymers. In such embodiments, each acrylic acid-acrylate copolymer is independently selected, and may be the same as or different from any other acrylic acid-acrylate copolymer.

The acrylic acid-acrylate copolymer may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent, an organic solvent, etc., such as those described above. When utilized, the carrier vehicle will be selected based on the particular components of the oral care composition and/or the miscibility component (B), such as the particular acrylic acid-acrylate copolymer selected, the pH of the composition and/or the miscibility component (B), etc. It will be appreciated that the acrylic acid-acrylate copolymer may be combined with the carrier vehicle, if utilized, prior to, during, or after being combined with any other components of the miscibility component (B). In certain embodiments, however, the miscibility component (B) is free from, alternatively substantially free from carrier vehicles.

The miscibility component (B) may be present in the composition in any amount, which will be selected by one of skill in the art, e.g. dependent upon the particular components (A), (B), and (C) selected, etc. Typically, the amount of miscibility component (B) is selected based on the concentration of the acrylic acid-acrylate copolymer therein. More specifically, the composition typically comprises an amount of the miscibility component (B) to comprise the acrylic acid-acrylate copolymer in an amount of from 8 to 12 wt. %, based on the total weight of the composition. In specific embodiments, the composition comprises the miscibility component (B), alternatively the acrylic acid-acrylate copolymer thereof, in an amount of from 8 to 10 wt. %, alternatively of from 8 to 9 wt. %, based on the total weight of the composition.

The plasticizer (C) comprises a polyalkylene glycol. In general, the polyalkylene glycol of the plasticizer (C) is a homopolymer or copolymer comprising repeating alkyl ether (i.e., oxyalkylene) subunits. Typically, as will be appreciated by those of skill in the art, the polyalkylene glycol is a homopolymer or copolymer of ethylene oxide, propylene oxide, and/or butylene oxide, etc., or the same as the reaction product of such alkylene oxides. As such the polyalkylene glycol may comprise, alternatively may be, a polyethylene glycol (PEG), a polypropylene glycol (PPG), a polybutylene glycol (PBG), etc., or a combination thereof (e.g. a PEG-PPG copolymer, a PEG-PPG-PBG triblock copolymer, etc.). When the polyalkylene glycol comprises more than one type of alkyl ether subunits (e.g. an ethylene glycol and a propylene glycol group), the subunits need not be in any particular order, but instead may be randomized or ordered (e.g. in one or more blocks, which may independently be ordered or random). In certain embodiments, the polyalkylene glycol comprises, alternatively is, a PEG homopolymer.

In certain embodiments, the polyalkylene glycol comprises the general formula R³(CₓH₂ₓO)_{y}R⁴, where each of R³ and R⁴ is independently selected from hydrocarbyl groups and H, each subscript x is independently from 2 to 5 in each moiety indicated by subscript y; and subscript y is from 4 to 20. Typically, of R³ and R⁴ is independently selected from H and hydrocarbyl groups. Suitable hydrocarbyl groups may generally be substituted or unsubstituted, saturated or unsaturated, linear, branched, or cyclic, or combinations thereof, and may include one or more alkyl groups, aryl groups, alkenyl groups, halocarbon groups, and the like, as well as derivatives, modifications, and combinations thereof. In certain embodiments, subscript y is from 4 to 16, such as from 4 to 12, alternatively from 6 to 12, alternatively from 8 to 10.

It is to be appreciated that the polyalkylene glycol may comprise other subunits and/or segments in additional to oxyalkylene and/or polyoxyalkylene subunits. Examples of such other subunits include those derived from alcohols, polyols (e.g. glycerols), carboxylic acids and/or esters, and the like, as well as derivatives, modifications, and combinations thereof, which may be repeating or non-repeating in the polyalkylene glycol, or otherwise appended thereto (e.g. as an end-cap).

Typically, the polyalkylene glycol comprises a molecular weight of from 150 to 800. For example, in certain embodiments the polyalkylene glycol comprises a molecular weight of from 150 to 600, alternatively from 200 to 600, alternatively from 250 to 600, alternatively from 250 to 550, alternatively from 250 to 500, alternatively from 300 to 500, alternatively from 350 to 450.

In certain embodiments, the plasticizer (C) comprises more than one polyalkylene glycol, such as 2, 3, 4, or more polyalkylene glycols. In such embodiments, each polyalkylene glycol is independently selected, and may be the same as or different from any other polyalkylene glycol.

The polyalkylene glycol may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent, an organic solvent, etc., such as those described above. When utilized, the carrier vehicle will be selected based on the particular components of the oral care composition and/or the plasticizer (C), such as the particular polyalkylene glycol selected. It will be appreciated that the polyalkylene glycol may be combined with the carrier vehicle, if utilized, prior to, during, or after being combined with any other components of the plasticizer (C). In certain embodiments, however, the plasticizer (C) is free from, alternatively substantially free from carrier vehicles.

The plasticizer (C) may be present in the composition in any amount, which will be selected by one of skill in the art, e.g. dependent upon the particular components (A), (B), and (C) selected, etc. Typically, the amount of plasticizer (C) is selected based on the concentration of the polyalkylene glycol therein. More specifically, the composition typically comprises an amount of the plasticizer (C) to comprise the polyalkylene glycol in an amount of from 20 to 30 wt. %, based on the total weight of the composition. In specific embodiments, the composition comprises the plasticizer (C), alternatively the polyalkylene glycol thereof, in an amount of from 20 to 25 wt. %, alternatively of from 20 to 23 wt. %, based on the total weight of the composition.

The properties of the composition may be controlled (e.g. tuned) by adjusting during formulation, as will be understood by those of skill in the art. For example, the adhesive strength of the composition can be controlled (i.e., increased or decreased) during formulation, e.g. by varying the relative amount of different components. However, to balance the processability and cohesiveness of the composition, and the flexibility, adhesiveness, and decreased brittleness of films prepared therewith, the composition is typically formulated to comprise a particular ratio of components (A), (B), and (C). For example, in certain embodiments, the composition is formulated to comprise a ratio of components (A) and (B) of from 4:1 to 6:1 A:B, such as from 4.5:1 to 5.5:1, alternatively of from 4.75:1 to 5.25:1, alternatively of 4.9:1 to 5.1:1 A:B. In these or other embodiments, the composition is formulated to comprise a ratio of components (A) and (C) of from 1:1 to 3:1 A:C, such as from 1.5:1 to 2.5:1, alternatively of from 1.75:1 to 2.25:1, alternatively of 1.9:1 to 2.1:1 A:C. In certain embodiments, the composition is formulated to comprise a ratio of components (B) and (C) of from 2:1 to 3:1 C:B, such as from 2.25:1 to 2.75:1, alternatively of from 2.4:1 to 2.6:1 C:B. In particular embodiments, the composition comprises components (A), (B), and (C) in a ratio of 5:1:2.5 A:B:C.

In certain embodiments, the composition further comprises (D) a whitening agent. In general, the whitening agent (D) may be referred to or otherwise described as a tooth whitening agent. As will be understood by those of skill in the art, the term “whitening agent” generally refers to a compound or composition capable of oxidizing, bleaching, and/or removing stains from a surface, such as a tooth. Accordingly, the whitening agent (D) comprises, alternatively is, a whitening compound. Typically, whitening compounds comprise and/or provide a peroxide, a chlorite, an enzyme capable of such action. Examples of suitable whitening compounds include peroxides, hypochlorite, chlorine dioxide, metal chlorites (e.g. calcium chlorite, barium chlorite, magnesium chlorite, lithium chlorite, sodium chlorite, potassium chlorite, etc.), perborates, percarbonates, and the like, as well as derivatives, modifications, and combinations thereof.

In certain embodiments, the whitening agent (D) is further defined as a peroxide whitening agent, i.e., a whitening agent comprising, alternatively consisting essentially of, alternatively consisting of, a peroxide-based whitening compound. Examples of peroxide whitening compounds include hydrogen peroxides, organic peroxides (e.g. carbamide peroxides), inorganic peroxides (e.g. sodium peroxides, calcium peroxides, etc.), peroxyacids (e.g. phthalimidoperoxyhexanoic acid, phthalimidoperoxycaproic acid, etc.), and the like, as well as derivatives, modifications, and combinations thereof. In certain embodiments, the whitening agent (D) comprises, alternatively is, hydrogen peroxide.

The whitening agent (D) may be utilized in any form, such as neat or disposed in a carrier vehicle, such as any of those described above. Likewise, the whitening agent (D) may be combined with the carrier vehicle, if utilized, prior to, during, or after being combined with any other components of the composition.

In certain embodiments, the whitening agent (D) comprises more than one whitening compound, such as 2, 3, 4, or more of the particular whitening compounds described herein. In such embodiments, each whitening compound is independently selected, and may be the same as or different from any other whitening compound in the whitening agent (D).

For example, the whitening agent (D) may comprise (i.e., in addition to a peroxide whitening agent or other whitening compound described above), a surfactant or detergent, an anti-tartar/anti-calculus compound, a tartar dissolving compound (e.g. such as a betaine, amine oxide, etc.) and the like, or combinations thereof. Particular examples of anti-tartar/anti-calculus compounds include phosphates such as pyrophosphates (e.g. dialkali metal pyrophosphate salts, tetra-alkali metal pyrophosphate salts, disodium dihydrogen pyrophosphates and hydrates thereof, tetrasodium pyrophosphate, tetrapotassium pyrophosphate, etc.), polyphosphates, polyphosphonates (e.g. ethane-1-hydroxy-1,1-diphosphonate, 1-azacyclolieptane-1,1-diphosphonate, linear alkyl diphosphonates, etc.) and salts thereof, linear carboxylic acids, and the like, as well as derivatives, modifications, and combinations thereof, and/or any of the additional examples of such compounds described below.

The whitening agent (D) may be present in the composition in any amount, which will be selected by one of skill in the art, e.g. dependent upon the particular whitening agent (D) selected (e.g. the particular whitening compound(s) therein). Typically, the composition comprises the whitening agent (D) in an amount of from 0.1 to 20 wt. %, based on the total weight of the composition. In specific embodiments, the composition comprises the whitening agent (D), alternatively the whitening compound(s), in an amount of from 5 to 20 wt. %, alternatively of from 5 to 15 wt. %, based on the total weight of the composition. In certain embodiments, the whitening agent (D) is hydrogen peroxide, and the composition comprises the hydrogen peroxide in an amount of from 5 to 15 wt. %, based on the total weight of the composition. In other embodiments, the whitening agent (D) is a carbamide peroxide, and the composition comprises the carbamide peroxide in an amount of from 15 to 45 wt. %, based on the total weight of the composition. In yet other embodiments, the whitening agent (D) is a phthalimidoperoxycaproic acid, and the composition comprises the phthalimidoperoxycaproic acid in an amount of from 0.1 to 5 wt. %, based on the total weight of the composition.

In some embodiments, the oral care composition comprises one or more additional components (i.e., additives), such as a flavoring agent, a surfactant, a thickener, a pH modifier, a buffer, a filler, a preservative, a coloring agent, a viscosity modifier, a chelator, an active agent, or combinations thereof.

In general, the composition is formulated based on a desired use thereof, further processing and/or compounding to be performed, etc. As such, the amount of each additive, when utilized, will be independently selected by those of skill in the art, e.g. in view of a desired end use of the composition, the nature/type of other additives to be utilized, the amounts of components (A), (B), (C), and/or (D) utilized, etc. Likewise, while particular amounts are described below, it is to be appreciated that amounts outside these ranges may also be utilized. For example, the amounts of certain additives (e.g. the pH modifier, the buffer, etc.) may be selected based on the particular composition being formulated, such as to reach a particular pH of the same, as will be understood by those of skill in the art.

In certain embodiments, the composition comprises the flavoring agent. As will be understood by those of skill in the art, the term “flavoring agent” refers generally to compounds selected to impart and/or alter flavor of the composition. As such, the flavoring agent is typically selected from sweeteners, flavorants/flavorings, and the like, or combinations thereof.

Examples of flavorants include certain plant extracts and compounds thereof, as well as synthetic versions and derivatives of such compounds. Specific examples of such flavorant compounds include wintergreen, peppermint, spearmint, menthol, cinnamon, vanillin, flavor oils and oleoresins, fruit-based flavorants (e.g. those from lemon, orange, etc.), spices, and the like, as well as combinations thereof. Examples of sweeteners generally include sugars, sugar substitutes, and sugar alcohols. Specific examples of sweeteners include neotame, saccharin, dextrose, sucrose, fructose, lactose, maltose, levulose, aspartame, sodium cyclamate, D-tryptophan, dihydrochalcones, acesulfame, aspartame, xylitol, and the like, as well as combinations thereof. In particular embodiments, the composition comprises sodium saccharin.

In certain embodiments, the flavoring agent comprises more than one flavorant, such as 2, 3, 4, or more of the particular flavorants described herein. In such embodiments, each flavorant is independently selected, and may be the same as or different from any other flavorant in the flavoring agent.

When utilized, the composition typically comprises the flavoring agent in an amount of from 0.1 to 5 wt. %, based on the total weight of the composition. In specific embodiments, the composition comprises the flavoring agent in an amount of from 0.2 to 0.5 wt. %, such as from 0.2 to 0.4, alternatively from 0.2 to 0.35, alternatively from 0.23 to 0.33 wt. %, based on the total weight of the composition.

In some embodiments, the composition comprises the surfactant. The surfactant is not particularly limited, and may comprise, alternatively may be, a nonionic surfactant, an anionic surfactant, a cationic surfactant, a zwitterionic surfactant, or a combination thereof.

Specific examples of nonionic surfactants include polyoxyalkylenes such as polyoxyethylenes (e.g. polyethylene and polypropylene glycols (i.e., PEGs and PPGs), including alkyl ethers, esters, and copolymers thereof, such as condensates with amines, amides, fatty esters, fatty alcohols, etc.), ethoxylated alcohols such as ethoxylated trimethylnonanols, and the like, as well as derivatives, modifications, and combinations thereof. Specific examples of anionic surfactants include salts and alkali metal soaps of fatty acids, sulfonates (e.g. alkyl benzene sulphonates, etc.), sulfates (e.g. sulfates of monoglycerides, esters, ethoxylated alcohols, succinates, etc.), phosphate esters, amino acid derivatives (e.g. N-acyl glutamates, N-acyl glycinates, etc.), anionic alkyl polyglycoside (e.g. acyl-D-galactoside urinate, etc.), and the like, as well as derivatives, modifications, and combinations thereof. Specific examples of amphoteric and/or zwitterionic surfactants include betaines (e.g. alkyl betaines, N-alkylamidobetaines, etc.), amino acids (e.g. alkylamino acid salts, alkyl polyaminocarboxylates, N-acylamino acids such as N-alkylaminoacetate and disodium cocoamphodiacetate, etc.), imidazolines, sultaines, alkyl amphoacetates, amine oxides (e.g. stearamine oxide, etc.), and the like, as well as derivatives, modifications, and combinations thereof. In particular embodiments, the composition comprises a polyoxyalkylene surfactant, such as a PEG-PPG copolymer. In some such embodiments, the surfactant comprises, alternatively is, a poloxamer (i.e., a PEG-PPG-PEG triblock copolymer), such as poloxamer 407.

In certain embodiments, the composition comprises more than one surfactant, such as 2, 3, 4, or more of the surfactants described herein. In such embodiments, each surfactants is independently selected, and may be the same as or different from any other surfactants in the composition.

When utilized, the composition typically comprises the surfactant in an amount of from 0.01 to 1 wt. %, based on the total weight of the composition. In specific embodiments, the composition comprises the surfactant in an amount of from 0.05 to 0.5 wt. %, such as from 0.05 to 0.25, alternatively from 0.05 to 0.15, alternatively from 0.07 to 0.11 wt. %, based on the total weight of the composition.

In certain embodiments, the composition comprises the thickener, which may comprise, alternatively may be, a natural or synthetic thickening compound, or combinations thereof. Examples of thickening compounds generally include gums (e.g. xanthan gum) collagen, galactomannans, starches, starch derivatives and hydrolysates, cellulose derivatives (e.g. methyl cellulose, hydroxypropyl cellulose, hydroxyethyl cellulose, hydroxypropyl methyl cellulose, etc.), colloidal silicic acids, polyvinyl alcohols, vinylpyrrolidone-vinylacetate-copolymers, polyethylene glycols, polypropylene glycols, and the like, as well as derivatives, modifications, and combinations thereof. In specific embodiments, the composition comprises a xanthan gum.

When utilized, the composition typically comprises the thickener in an amount of from 0.01 to 1 wt. %, based on the total weight of the composition. In specific embodiments, the composition comprises the thickener in an amount of from 0.05 to 0.5 wt. %, such as from 0.05 to 0.4, alternatively from 0.05 to 0.3, alternatively from 0.05 to 0.2 wt. %, based on the total weight of the composition.

In certain embodiments, the composition comprises the pH modifier. The pH modifier is not particular limited, and may comprise or be any compound suitable for modifying the pH of the composition (e.g. during formulation, etc.). As will be understood by those of skill in the art, the pH modifier typically comprises, alternatively is, an acid or a base.

Examples of acids suitable for use in or as the pH modifier include mineral acids (e.g. hydrochloric acid, phosphoric acid, sulfuric acid, etc.), organic acids (e.g. citric acid, etc.), and the like, as well as derivatives, modifications, and combinations thereof. Examples of bases suitable for use in or as the pH modifier include alkali metal hydroxides (e.g. sodium hydroxide, potassium hydroxide, etc.), carbonates (e.g. alkali metal carbonate salts such as sodium carbonate, etc.), phosphates, and the like, as well as derivatives, modifications, and combinations thereof. In particular embodiments, the pH modifier is sodium hydroxide.

In certain embodiments, the composition comprises more than one pH modifier, such as 2, 3, 4, or more of the pH modifiers described herein. In such embodiments, each pH modifier is independently selected, and may be the same as or different from any other pH modifier in the composition.

When utilized, the composition typically comprises the pH modifier in an amount of from 0.005 to 0.5 wt. %, based on the total weight of the composition. In specific embodiments, the composition comprises the pH modifier in an amount of from 0.005 to 0.1 wt. %, such as from 0.01 to 0.1, alternatively from 0.01 to 0.075, alternatively from 0.01 to 0.05 wt. %, alternatively from 0.01 to 0.04, alternatively from 0.01 to 0.037 wt. %, based on the total weight of the composition.

In certain embodiments, the composition comprises the buffer. The buffer is not limited, and may comprise, alternatively may be, any buffering compound capable of adjusting the pH of the composition and/or maintaining (e.g. regulating) the pH of the composition in a particular range. As will be understood by those of skill in the art, the buffer is generally selected to impart the composition with a pH compatible in an oral environment, e.g. such that the composition will not leach minerals from the surface of teeth (i.e., via demineralization). As will also be understood by those of skill in the art, examples of suitable buffers and buffering compounds may overlap with certain pH modifiers, including those described above, due to the overlap in functions between the additives. As such, when utilized, the buffer and the pH modifier may be independently or collectively selected in view of each other.

In general, the buffer comprises a buffering compound that is an acid, a base, or a salt (e.g. comprising the conjugate base/acid of an acid/base), and may comprise a calcium and/or fluoride salt. Examples of buffering compounds generally include alkali metal hydroxides (e.g. sodium hydroxide, potassium hydroxide, etc.), carbonates (e.g. sesquicarbonates, alkali metal carbonate salts such as sodium carbonate, etc.), borates, silicates, phosphates, imidazoles, citric acid, sodium citrate, and the like, as well as derivatives, modifications, and combinations thereof. In certain embodiments, the buffer is selected from citrate buffers, glycerol buffers, borate buffers, phosphate buffers, and combinations thereof (e.g. citric acid-phosphate buffers, etc.). In particular embodiments, the buffer comprises, alternatively is, an ethylenediaminetetraacetic acid (EDTA) buffer, such as disodium EDTA.

In certain embodiments, the buffer and/or the composition comprises more than one buffering compound, such as 2, 3, 4, or more of the buffering compounds described herein. In such embodiments, each buffering compound is independently selected, and may be the same as or different from any other buffering compound in the buffer and/or composition.

When utilized, the composition typically comprises the buffer in an amount of from 0.01 to 1 wt. %, based on the total weight of the composition. In specific embodiments, the composition comprises the buffer in an amount of from 0.05 to 0.75 wt. %, such as from 0.05 to 0.7, alternatively from 0.05 to 0.5, alternatively from 0.05 to 0.25, alternatively from 0.05 to 0.2, alternatively from 0.06 to 0.2 wt. %, based on the total weight of the composition.

In certain embodiments, the composition comprises the filler. Suitable fillers may be absorbent or non-absorbent, inert, expansive, etc., or combinations thereof. Specific examples of fillers include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, clays such as laponite, polyethylenes, polypropylenes, polyurethane-polyether-amide copolymers, polyesters and polyester copolymers, nylons, and the like, as well as derivatives, modifications, and combinations thereof.

When utilized, the composition typically comprises the filler in an amount of from 0.01 to 5 wt. %, based on the total weight of the composition. In specific embodiments, the composition comprises the filler in an amount of from 0.05 to 2.5 wt. %, such as from 0.1 to 1 wt. %, based on the total weight of the composition.

In some embodiments, the composition comprises the preservative. The preservative is not generally limited, and may comprise, alternatively may be, any preservative known in the art. Examples of preservatives include phenols, benzoic acids and benzoates, etc. Specific examples of preservatives include phenylethyl alcohols, phenoxyethyl alcohols, chlorobutanols, 4-hydroxybenzoic acids (e.g. 4-hydroxybenzoic acid methylester, propylester etc.), benzalkonium chlorides, p-chlorocresols, cetylpyridinium chlorides, chlorohexidines (e.g. chlorohexidine diacetate, gluconate, etc.), ethanol, propylene glycol, and the like, as well as derivatives, modifications, and combinations thereof.

When utilized, the composition typically comprises the preservative in an amount of from 0.01 to 5 wt. %, based on the total weight of the composition. In specific embodiments, the composition comprises the preservative in an amount of from 0.05 to 2.5 wt. %, such as from 0.1 to 1 wt. %, based on the total weight of the composition.

In certain embodiments, the composition comprises the coloring agent. The coloring agent is not particularly limited, and may be utilized in any form (e.g. powdered, granulated, liquid, emulsified, suspended, dissolved/solvated, etc.). Likewise, the coloring agent may comprise, alternatively may be, a dye, a pigment, and/or any other such substance capable of imparting a color to the composition (collectively, “colorizing compounds”).

Suitable colorizing compounds for use in and/or as the coloring agent include those derived from natural sources (e.g. vegetables, minerals, or animals, etc.) as well as synthetic colorizing compounds. Particular examples of suitable colorizing compounds include those conventionally used with foods, supplements, drugs, medical devices, etc., such as sodium salts of tetraiodofluorescein (e.g. FD&C Red No. 3), disodium salts of 6-hydroxy-5-{(2-methoxy-5-methyl-4-sulphophenyl)azo}-2-naphthalenesulfonic acid (e.g. Food Red 17), sodium salts of mono- and disulfonic acids of quinophthalone and/or 2-(2-quinolyl)indanedione (e.g. Food Yellow 13), sodium salts of 4-p-sulfophenylazo-1-p-sulfophenyl-5-hydroxypyrazole-3 carboxylic acid (e.g. FD&C Yellow No. 5), sodium salt of p-sulfophenylazo-B-napthol-6-monosulfonate (e.g. FD&C Yellow No. 6), disodium salts of 4-{[4-(N-ethyl-p-sulfobenzylamino)-phenyl]-(4-hydrox-2-sulfonium-phenyl)-methylene}-[1-(N-ethyl-N-p-sulfobenzyl)-3,5-cyclohexadienimine] (e.g. FD&C Green No. 3), disodium salts of dibenzyldiethyl-diaminotriphenylcarbinol trisulfonic acid anhydrite (e.g. FD&C Blue No. 1), sodium salts of disulfonic acid of indigotin (e.g. FD&C Blue No. 2), Allura Red AC (i.e., FD&C Red No. 40), Orange B, 1-(2,5-Dimethoxy-phenylazo)-naphthalen-2-ol (i.e., Citrus Red No. 2), and the like, as well as derivatives, modifications, and combinations thereof. Additional examples of suitable colorizing compounds include various extracts (e.g. annatto extracts, cochineal extracts such as carmine, grape color extracts, grape skin extracts such as enocianina, etc.), carotenoids (e.g. beta-carotene, beta-apo-8′-carotenal, canthaxanthin, etc.), powders of fruits, vegetables, and other plants (e.g. beet powder, spices such as paprika, saffron, turmeric, etc.) and extracts thereof (e.g. paprika oleoresin, turmeric oleoresin, etc.), fruit and vegetable juices and oils (e.g. carrot oil, etc.), caramel color, riboflavin, ferrous gluconate, cottonseed flours (e.g. those processed via toasting, defatting or partially defatting, cooking, etc.), non-toxic inorganic water-insoluble pigments (e.g. titanium dioxide, chromium oxide greens, ultramarine blues and pinks, and ferric oxides, etc.), and the like, as well as derivatives, modifications, and combinations thereof.

In certain embodiments, the coloring agent comprises more than one colorizing compound, such as 2, 3, 4, or more of the particular colorizing compounds described herein. In such embodiments, each colorizing compound is independently selected, and may be the same as or different from any other colorizing compound in the coloring agent.

When utilized, the composition typically comprises the coloring agent in an amount of from 0.01 to 5 wt. %, based on the total weight of the composition. In specific embodiments, the composition comprises the coloring agent in an amount of from 0.05 to 2.5 wt. %, such as from 0.1 to 1 wt. %, based on the total weight of the composition.

In certain embodiments, the composition comprises the viscosity modifier. The viscosity modifier is not limited, and may comprise, alternatively may be, any viscosity/rheology modifier/additive known in the art. General examples of such viscosity modifiers include waxes (e.g. paraffin waxes, microcrystalline waxes, polyamide waxes, etc.), polymers (e.g. polyamides), oils (e.g. castor oil and derivatives thereof, such as hydrogenated castor oil, etc.), metal soaps (e.g. calcium, aluminum, and/or barium stearates, etc.), and the like, as well as derivatives, modifications, and combinations thereof. However, as will be understood by those of skill in the art, the particular viscosity modifier utilized will be selected in view of the composition, e.g. to facilitate compounding, incorporating of fillers, de-airing, and/or mixing of the composition (e.g. during preparation thereof).

When utilized, the composition typically comprises the viscosity modifier in an amount of from 0.01 to 5 wt. %, based on the total weight of the composition. In specific embodiments, the composition comprises the viscosity modifier in an amount of from 0.01 to 1 wt. %, such as from 0.05 to 1, alternatively from 0.05 to 0.75, alternatively from 0.05 to 0.5 wt. %, based on the total weight of the composition.

In some embodiments, the composition comprises the chelator. The chelator is not limited, and may comprise, alternatively may be, any compound capable of chelating and/or sequestering one or more metals (e.g. metal ions) known in the art. General examples of such chelators include monodentate and polydentate chelating compounds, as well as mixtures thereof, including alkylene polyamines (e.g. ethylenediamines, diethylentriamines), polycarboxyl compounds (e.g. citric acid, citrates, oxalic acid etc.), amino acids (e.g. am inopolycarboxylic acids, including ethylenediamine succinic acids (e.g. EDDS), acetic acids (e.g. EDTA), glutamic acid etc.), hydroxamic acids, and the like, as well as derivatives, modifications (e.g. conjugate acid/bases, salts, etc.), and combinations thereof. As will be understood by those of skill in the art, examples of suitable chelators may overlap with certain pH modifiers and/or buffering compounds, including those described above, due to the overlap in functions between the additives. As such, when utilized, the chelator, buffer, and/or pH modifier may be independently or collectively selected in view of each other. In particular embodiments, the chelator comprises, alternatively is, an ethylenediaminetetraacetic acid (EDTA), such as disodium EDTA.

When utilized, the composition typically comprises the chelator in an amount of from 0.01 to 1 wt. %, based on the total weight of the composition. In specific embodiments, the composition comprises the chelator in an amount of from 0.05 to 0.75 wt. %, such as from 0.05 to 0.7, alternatively from 0.05 to 0.5, alternatively from 0.05 to 0.25, alternatively from 0.05 to 0.2, alternatively from 0.06 to 0.2 wt. %, based on the total weight of the composition.

In certain embodiments, the composition comprises the active agent. The active agent is not limited, and may comprise, alternatively may be, any compound capable of exhibiting a desired activity (e.g. a therapeutic activity, prophylactic activity, ameliorative activity, cosmetic activity, etc.) known in the art. As will be understood by those of skill in the art, examples of suitable active agents may be designated or otherwise referred to as “oral care agents,” “pharmaceutical agents,” therapeutic agents,” etc., or combinations thereof (i.e., “pharmaceutical oral care agents”), and are typically useful in treating, ameliorating, and/or preventing a physiological conditions involving teeth and/or surrounding tissues.

Examples of compounds suitable for use in or as the active agent generally include anti-cavity and/or anti-decay agents, anti-plaque, anti-tartar, and/or anti-calculus agents, anti-inflammatory agents (e.g. non-steroidal anti-inflammatories, steroidal anti-inflammatories, etc.) and/or analgesics, anesthetics, tooth desensitizing agents, antimicrobial agents, stimulants (e.g. nicotine, caffeine, etc.), nutritional supplements, cosmetic active agents (e.g. fragrances, breath fresheners, etc.), and the like, as well as derivatives, modifications, and combinations thereof.

Examples of anti-cavity and/or anti-decay agents generally include fluoride and fluoride-containing/producing compounds, such as sodium fluoride, potassium fluoride, ammonium fluoride, and the like, as well as derivatives and combinations thereof.

Examples of anti-plaque, anti-tartar, and/or anti-calculus agents generally include compounds that inhibit, prevent, or reduce the formation and/or accumulation of plaque, tartar, calculus, and/or dental caries, such as phosphates (e.g. pyrophosphates, polyphosphates, polyphosphonates, etc.), carboxylic acids and/or carboxylates (e.g. linear carboxylic acids, polycarboxylates, etc.), as well as derivatives, salts, and combinations thereof. Some specific examples of these include alkyl diphosphonates (e.g. ethane-1-hydroxy-1,1-diphosphonate, 1-azacycloheptane-1,1-diphosphonate, etc.), alkali metal pyrophosphate salts (e.g. di- and tetra-sodium and/or potassium salts, etc., and hydrates thereof, including hydrated and unhydrated forms of disodium dihydrogen pyrophosphate, tetrasodium pyrophosphate, tetrapotassium pyrophosphate, etc.), sodium zinc citrate, and the like, as well as derivatives, modifications, and combinations thereof. Other examples include enzymes, such as proteases, lipases, dextranases, glucanohydrolases, endoglycosidases, mucinases, amylases, mutanases, etc., as well as combinations thereof, which will be selected based on compatibility with each other enzyme in a mixture prepared therefrom, as understood by those of skill in the art.

Examples of non-steroidal anti-inflammatories (i.e., NSAIDs)/analgesic agents include acetylsalicylic acids (i.e., aspirin), methyl salicylates, monoglycol salicylates, acetaminophen, isobutylphenylpropionic acids (e.g. ibuprofen), flurbiprofens, mefenamic acids, flufenamic acids, indomethacins, diclofenacs (e.g. diclofenac, diclofenac sodium, etc.), alclofenacs, fentizacs, bufexamacs, piroxicams, phenylbutazones, oxyphenbutazones, clofezones, pentazocines, mepirizoles, tiaramides (e.g. tiaramide hydrochloride), and the like, as well as combinations thereof.

Examples of steroidal anti-inflammatories include hydrocortisones (e.g. hydrocortisone, hydrocortisone acetate, etc.), prednisolones (e.g. prednisolone, prednisolone acetate, methylprednisolone, etc.), dexamethasones, triamcinolone acetonides, fluocinolone acetonides, methasones (e.g. dexamethasone acetate, betamethasone, betamethasone valerate, beclomethasone dipropionate, etc.), flumetasones, fluorometholones, budesonides, and the like, as well as combinations thereof.

Examples of anesthetics include lidocaines (e.g. lidocaine, lidocaine hydrochloride, etc.), procaines (e.g. procaine, chloroprocaine, oxyprocaine, hydrochlorides and other salts thereof, etc.), dibucaines (e.g. dibucaine, dibucaine hydrochloride, etc.), benzocaines, tetracaines (e.g. tetracaine, tetracaine hydrochloride, etc.), mepivacaine, piperocaine, cocaines, butylaminobenzoic acid 2-(diethylamino) ethyl esters (e.g. p-butylaminobenzoic acid 2-(diethylamino) ethyl ester hydrochloride), and the like, as well as derivatives, modifications, and combinations thereof.

Examples of tooth desensitizing agents generally include compounds suitable for treating, preventing, reducing, and/or ameliorating unpleasant and/or painful sensations in dentin caused by thermal, evaporative, tactile, osmotic, and/or chemical stimuli. Specific examples of such compounds include certain metal salts (e.g. potassium nitrate, strontium chloride, potassium oxalates, etc.), glutaraldehydes, acidulated phosphate fluoride, and the like, as well as derivatives, modifications, and combinations thereof.

Suitable antimicrobial agents generally include antibiotics, bactericides, disinfectants, and fungicides, as well as combinations thereof. Examples of antibiotics include penicillins, tetracyclines (e.g. chlortetracycline, oxytetracycline, etc.), erythromycins, streptomycins, bacitracins, meticillins, oxacillins, cefalotins, cefaloridins, lincomycins, metacyclines, chloramphenicols, kanamycins, gentamicins, cycloserines, and the like, as well as derivatives, modifications, and combinations thereof. Examples of bactericides/disinfectants include phenol, chlorhexidine, iodopovidone, thimerosal, thymol, eugenol, benzalkonium chloride, benzethonium chloride, cetylpyridinium chloride, trimethylammonium bromide, zinc pyrithione, and the like, as well as derivatives, modifications, and combinations thereof. Examples of fungicides include antifungal imidazole compounds (e.g. econazoles including econazole, econazole nitrate, etc., itraconazole, fluconazole, clotrimazole, ketoconazole, miconazole, etc.), antifungal undecenoates (e.g. undecenoic acid, zinc undecenoate, etc.), amphotericin, griseofulvin, nystatin, terbinafines (e.g. terbinafine, terbinafine hydrochloride, etc.), and the like, as well as derivatives, modifications, and combinations thereof.

Suitable nutritional supplements generally include vitamins such as vitamin A (i.e., retinoids, including retinol, retinal, retinyl esters, and retinoic acid), vitamin B1 (i.e., thiamine), vitamin B2 (i.e., riboflavin), vitamin B3 (i.e., niacin), vitamin B5 (i.e., pantothenic acid), vitamin B6 (i.e., pyridoxine), vitamin B9 (i.e., folic acid), vitamin B12 (i.e., cobalamin), vitamin H (i.e., biotin), vitamin C (i.e., ascorbic acid), vitamin D (e.g. calciferol (i.e., vitamin D3), ergocalciferol (i.e., vitamin D2)), vitamin E (i.e., tocopherol), and vitamin K (e.g. phytomenadione (i.e., vitamin K1), menaquinone (i.e., vitamin K2)), minerals such as calcium, phosphorus, potassium, magnesium, sodium chloride, iron, zinc, copper, chromium, fluoride, iodine, selenium, manganese, and molybdenum, and combinations thereof.

When utilized, the composition typically comprises the active agent in an amount sufficient to elicit a desired physiological effect when disposed on the teeth and/or surrounding tissues of a user. In general, the composition comprises the active agent in an amount of from 0.0001 to 10 wt. %, based on the total weight of the composition. In specific embodiments, the composition comprises the active agent in an amount of from 1 to 10 wt. %, such as from 1 to 9, alternatively from 1 to 8, alternatively from 1 to 7, alternatively from 1 to 6, alternatively from 1 to 5 wt. %, based on the total weight of the composition. In some embodiments, the composition comprises the active agent in an amount of from 0.0001 to 3 wt. %, such as from 0.0001 to 2, alternatively from 0.0001 to 1, alternatively from 0.001 to 1, alternatively from 0.001 to 0.5, alternatively from 0.01 to 0.5 wt. %, based on the total weight of the composition. One of skill in the art will appreciate that amounts outside the ranges above may be utilized as well. For example, in certain embodiment the active agent comprises, alternatively is, a fluoride anti-cavity/anti-decay compound, and the composition comprises the fluoride anti-cavity/anti-decay compound in an amount of from 50 to 10,000 ppm.

In certain embodiments, the composition comprises more than one active agent, such as 2, 3, 4, or more of the active agents described herein. In such embodiments, each active agent is independently selected, and may be the same as or different from any other active agent in composition.

Each additive may be utilized in any form, such as neat (i.e., absent solvents, carrier vehicles, diluents, etc.), or disposed in a carrier vehicle, such as those described above (e.g. selected based on the particular additive, the other components of the oral care composition, etc.). It will be appreciated that any one additive may be combined with the carrier vehicle, if utilized, prior to, during, or after being combined with any other components of the composition. Likewise, each additive may be combined with any other additive or component of the composition, prior to, during, or after being combined with any other components of the composition. For example, in certain embodiments, one or more of the additives are combined with the whitening agent (D) to form a whitening composition, which is then combined with a combination of components (A), (B), and (C).

The oral care composition is suitable for use in various applications, including in an oral care device. As such, an oral care device comprising the composition (the “device”) and a method for preparing the same are also provided.

In general, the oral care device comprises an oral care layer comprising and/or formed from the oral care composition described above. Typically, the oral care composition is formulated to be processed into a film or sheet, e.g. via extrusion, solution casting, etc. As such, the oral care layer is typically prepared by melt extruding or solution casting the oral care composition and selected from extruded films/sheets and solvent-cast films/sheets.

In certain embodiments, the oral care layer is prepared by melt extrusion. In such embodiments, the components of the oral care composition (e.g. components (A), (B), and (C)) are combined and then extruded to form the oral care layer as a film/sheet. In such embodiments, components (A), (B), and (C) may be combined in any order, optionally in the presence of a carrier vehicle, in a reaction vessel, mixer, in a chamber of an extruder, etc. For example, the components (A), (B), and (C) may be combined together via blending/mixing (e.g. via a Brabender mixer or Baker Perkins Blender), and subsequently extruded into a film/sheet (e.g. via a single or twin-screw extruder). Alternatively, the composition may be pelletized after blending, and subsequently melt-extruded. As will be understood by those of skill in the art, blending and/or extruding the composition may be carried out at room temperature, or at an elevated temperature (e.g. from greater than ambient temperature to 200° C., such as from 30 to 200, alternatively from 50 to 200, alternatively from 75 to 200, alternatively from 95 to 200, alternatively from 100 to 195° C. Likewise, it is to be appreciated that multiple temperatures may be independently selected and utilized sequentially (e.g. in a temperature ramp) and/or concurrently (e.g. in a multi-zone extruder comprising two more zones of different temperatures.

Typically, the oral care layer comprises an average thickness of from 3 to 20 mil (i.e., 0.076-0.508 mm), such as from 3 to 15 mil (i.e., 0.076-0.381 mm), alternatively from 4 to 12 mil (i.e., 0.102-0.305 mm). In certain embodiments, the oral care layer comprises an average thickness of from 4 to 6 mil (i.e., 0.102-0.152 mm), alternatively of 5 mil (i.e., 0.127 mm). In particular embodiments, the oral care layer comprises an average thickness of from 9 to 11 mil (i.e., 0.229-0.279 mm), alternatively of 10 mil (i.e., 0.254 mm).

In certain embodiments, extruding or solution casting the oral care composition prepares an inactive oral care layer, which is then treated with the whitening agent (D) to give an active oral care layer. In such embodiments, the inactive oral care layer typically comprises, alternatively consists essentially of, components (A), (B), and (C). In some such embodiments, the inactive oral care layer is treated with the whitening composition described above, i.e., a combination of the whitening agent (D), one or more additives, and optionally in a carrier vehicle. In such embodiments, the final formulation of the active oral care layer will correspond to the ratios and amounts described above with respect to each component utilized. In some embodiments, the active oral care layer comprises an average thickness greater than the inactive oral care layer, e.g. due to swelling caused by the whitening composition. In such embodiments, the inactive oral care layer typically comprises an average thickness in a lower portion of the ranges described above (e.g. from 3 to 6 mil (i.e., 0.076-0.152 mm), 5 to 10 mil (i.e., 0.127-0.254 mm), etc.) and the active oral care layer comprises an average thickness in a higher portion of the ranges described above (e.g. from 5 to 10 mil (i.e., 0.127-0.254 mm), 10 to 20 mil (i.e., 0.254-0.508 mm), etc.).

The formulation of the oral care composition allows for preparation of the inactive oral care layer (e.g. via extrusion), which may be kept or otherwise stored for a period of time prior to forming the active oral care layer therewith. More specifically, the oral care composition provides the extruded oral care layer with improved shelf stability, as well as increased flexibility in film. In particular, the flexibility of the inactive oral care layer due to the oral care composition allowed for post-extrusion rolling (e.g. to prepare the inactive oral care layer as a rollstock) without breaking, shatter, or cracking, allowing for increased processability, easier storage, and increased convenience.

In certain embodiments, the oral care device comprises one or more additional layers (e.g. aside from the oral care layer), such that the oral care device may be defined as a composite or laminate. For example, in some such embodiments, the oral care device comprises a substrate, a release liner, or both, as described in further detail below.

In certain embodiments, the oral care device comprises a substrate. Typically, the substrate is disposed adjacent to the oral care layer. For example, in certain embodiments, the oral care layer is laminated with the substrate, such that the oral care device comprises a laminate including the substrate disposed adjacent the oral care layer.

In general, the substrate is a supportive/structural element of the oral care device, and is thus selected to provide support and or rigidity to the oral care device. Additionally, the substrate is selected to retain the components of the oral care layer and, in particular, the active oral care layer, to reduce or prevent migration of components of the oral care composition from one side of the oral care layer (i.e., by acting as a physical barrier). As such, the substrate is generally selected to be inert with regard to the oral care composition.

The dimensions (e.g. thickness) of the substrate are not particularly limited. For example, in general, the substrate may comprise an average thickness of from 5 to 25 mil (i.e., 0.127-0.635 mm), alternatively 10 to 25 mil (i.e., 0.254-0.635 mm), such as from 10 to 15 mil (i.e., 0.254-0.381 mm) or from 15 to 20 mil (i.e., 0.381-0.508 mm).

The substrate itself need not be a single, homogenous material, but may instead itself be a composite/laminate comprising multiple layers. Typically, the substrate comprises a structural layer and a wax layer. In certain embodiments, the substrate further comprises a backing layer. Each of these layers is described in further detail below.

In some embodiments, the substrate comprises the structural layer. Typically, the structural layer is disposed adjacent to and in contact with the oral care layer. As such, the structural layer is selected to be cohesive with the oral care layer and, in particular, components (A), (B), and (C) of the oral care composition, and is not otherwise limited. As will be understood by those of skill in the art, the structural layer may comprise, alternatively may be, any material suitable for providing structural integrity (e.g. mechanical strength/support) to the substrate, and thus the oral care device. Examples of such materials include non-wovens, wovens, synthetic films (e.g. perforated/apertured films), meshes, netting, and the like.

As will be understood by those of skill in the art, the term “woven” is used herein to designate and/or describe a material composed of textile fibers that are processed on conventional spindles, looms, knitting machines, etc., such as clothes, gauzes, felts, and the like. Comparatively, the term “non-woven” is used herein to designate and/or describe a material composed of textile fibers that are not processed on conventional spindles, looms, or knitting machines, but are instead chemically and/or mechanically bound/fused together. Particular examples of suitable non-woven materials for use in/as the structural layer include spun bonded fabrics (i.e., those formed by filaments that have been extruded, drawn, then laid on a continuous belt), spun-laced fabrics (i.e., those formed by entangling fibers in a repeating pattern, without binders), carded fabrics (i.e., those formed by a process similar to carding wool), “hook” or “loop” type fabrics (i.e., those used in hook and loop-type fasteners), and the like, as well as derivatives, modifications, and combinations thereof. Examples of suitable textile fibers include natural fibers (e.g. wool, etc.), synthetic fibers (e.g. fibers of polyolefins (e.g. polyethylenes, polypropylenes, etc.), polyesters, polyurethanes, polyamides, polyaramides, and/or glass), and combinations thereof.

The structural layer is selected to comprise an average thickness providing a flexural stiffness low enough to not substantially resist deformation, a resistance to shrinking to prevent delamination from the oral care layer, and a porosity/texture sufficient to allow for adhesion to the oral care layer, as will be understood by those of skill in the art. In certain embodiments, the structural layer comprises a non-woven material having a weight of from 15 to 40 gram per square meter (gsm), such as from 15 to 35, alternatively of from 20 to 35, alternatively of from 20 to 30, alternatively of from 22 to 28 gsm. In these or other embodiments, the structural layer comprises a woven material and/or a synthetic film having a weight substantially within one of these ranges and/or an average thickness corresponding to the average thickness of a non-woven material of such a weight.

In certain embodiments, the substrate comprises the wax layer. In general, the wax layer is disposed adjacent to and in contact with the structural layer (i.e., opposite the oral care layer).

The wax layer comprises, alternatively consists essentially of, a wax. General examples of suitable waxes include microcrystalline waxes (i.e., medium to high-molecular weight waxes comprising primarily branched-chain and/or cyclic hydrocarbons, with melting points of from ˜60-100° C.), paraffin waxes (i.e., low-molecular weight waxes comprising primarily straight-chain hydrocarbons, with melting points of ˜45-75° C.), plant and animal waxes (e.g. beeswax, carnauba wax, ouricury wax, candelilla wax, etc.), and the like, as well as derivatives, modifications, and combinations thereof. Specific examples of such waxes are known in the art and may be prepared via known methods or otherwise obtained (e.g. from numerous commercial suppliers).

The dimensions (e.g. thickness) of the wax layer are not particularly limited. For example, in general, the wax layer may comprise an average thickness of from 10 to 25 mil (i.e., 0.254-0.635 mm). In some embodiments, such as when the backing layer is also included in the substrate, the wax layer may comprise a thickness at the lower end of this range, such as from 10 to 15 mil (i.e., 0.254-0.381 mm), alternatively of from 11 to 13 mil (i.e., 0.279-0.330 mm). In other embodiments, such as when the substrate includes only the structural layer and wax layer, the wax layer may comprise a greater thickness, such as from 15 to 20 mil (i.e., 0.381-0.508 mm), alternatively of from 16 to 18 mil (i.e., 0.406-0.457 mm).

As introduced above, in certain embodiments, the substrate comprises the backing layer. When utilized, the backing layer is disposed adjacent and in contact with the wax layer (i.e., opposite the structural layer), such that the backing layer is the outer-most layer of the substrate.

Typically, the backing layer comprises a hydrocarbon resin. Examples of suitable resins include polyolefins (e.g. polyethylenes, polypropylenes, etc.), polyesters, polyurethanes, polyamides, polyaramides, and the like, as well as derivatives, modifications, and combinations thereof. One of skill in the art will readily appreciate that the backing layer and the structural layer are independently selected and, while may comprise the same resin, may be different in form.

The backing layer may be included as a structural element of the substrate (e.g. to provide physical rigidity, etc.), or as an aesthetic/comfort element. For example, the backing layer may be colored (i.e., may comprise a colorant) for marking, aesthetic appeal, etc. Likewise, the backing layer may comprise a flavoring agent to impart a taste to the substrate. The backing layer may also comprise decorative items, such beads, rhinestones, etc., and/or display symbols or images designed to be pleasing or attractive to a consumer.

The dimensions (e.g. thickness) of the wax layer are not particularly limited, and will be selected by those of skill in the art, e.g. based on the desired function of the backing layer, the inclusion of decorative items therein, etc. In certain embodiments, the backing layer comprises an average thickness of from 0.2 to 1 mil (i.e., 0.005-0.025 mm), such as from 0.2 to 0.6 mil (i.e., 0.005-0.015 mm), alternatively of 0.4 mil (i.e., 0.010 mm).

As introduced above, in some embodiments, the oral care device comprises a release liner. The release liner is disposed adjacent and in contact with the oral care layer, i.e., opposite the substrate. For example, in certain embodiments, the oral care layer is laminated with the release liner and the substrate, such that the oral care device comprises a laminate including the oral care layer disposed between the release liner and the substrate. As will be understood by those of skill in the art, the release liner is a disposable element of the oral care device selected to prevent exposure of the oral care layer prior to application, e.g. to protect the oral care composition and prevent premature contact with the same. As such, the release liner is selected to be generally inert with respect to the components of the oral care layer (e.g. the oral care composition, the whitening composition, etc.), other than being temporarily bound/bonded/adhered thereto, and also easily delaminated from the oral care layer.

Typically, the release liner comprises a rigid sheet of material that is optionally treated with a release coating. Examples of suitable materials generally include hydrocarbon resins (e.g. polyethylenes, polyesters, polyethylene terephthalates, etc.), papers, and the like. Release coatings are known in the art, and generally include non-stick compounds and/or compositions comprising fluoropolymers, silicones, waxes, etc.

The dimensions of the release liner are not particularly limited, and will be selected by those of skill in the art. For example, the release liner may be substantially the same size and shape (e.g. length and width) as the surface of the oral care layer on which it is disposed, or larger than the surface of the oral care layer to provide extra material for a user to handle for separating the release liner from the oral care layer. The thickness of the release layer is not particularly limited. However, in certain embodiments, the thickness of the release liner is selected based on intended processing to be performed on the oral care device, e.g. by tuning the thickness of the release liner to alter the flexibility and/or rigidity thereof. For example, in some embodiments, the release liner comprises an average thickness of from 0.5 to 2 mil (i.e., 0.0127-051 mm), such as from 1 to 2 mil (i.e., 0.025-051 mm), alternatively 1.5 mil (i.e., 0.038 mm). In other embodiments, the release liner comprises an average thickness of from 2 to 4 mil (i.e., 0.51-0.102 mm), such as 3 mil (i.e., 0.076 mm).

As will be appreciated in view of the description above, the oral care device may comprise various combinations of layers in addition to the oral care layer. In general, however, the oral care device typically comprises a laminate of the oral care layer, the substrate, and the release liner, with the oral layer disposed between the substrate and the release liner.

In some embodiments, the substrate is a two-layer composite including the structural layer adjacent the oral care layer, and the wax layer disposed adjacent the structural layer opposite the oral care layer. In such embodiments, a relatively thick wax layer (e.g. having a thickness of from 16 to 18 mil (i.e., 0.406-0.457 mm)) and a relatively thin release liner (e.g. having a thickness of from 1 to 2 mil (i.e., 0.025-051 mm)) are typically utilized. Moreover, in such embodiments, a relatively thick oral care layer (e.g. having a thickness of from 9 to 11 mil (i.e., 0.229-0.279 mm)) is utilized.

In other embodiments, the substrate is a three-layer composite including the structural layer adjacent the oral care layer, the backing layer disposed opposite oral care layer about the structural layer, and the wax layer disposed adjacent the structural layer and between the backing layer and the structural layer. In such embodiments, a relatively thin wax layer (e.g. having a thickness of from 11 to 13 mil (i.e., 0.279-0.330 mm) and a relatively thick release liner (e.g. having a thickness of from 2 to 4 mil (i.e., 0.51-0.102 mm)) are typically utilized. Moreover, in such embodiments, a relatively thin oral care layer (e.g. having a thickness of from 4 to 6 mil (i.e., 0.102-0.152 mm)) is utilized.

As will be appreciated from the description above, the method of preparing the oral care device includes preparing an oral care layer from the oral care composition. In certain embodiments, the method includes extruding the oral care composition to give the oral care layer. In some such embodiments, extruding the oral care composition prepares the inactive oral care layer, and the method further includes coating the inactive oral care layer with the whitening composition to give the oral care layer. As such, one of skill in the art will readily appreciate that the oral care layer may be prepared in a single-step or multi-step process, e.g. utilizing any of the compositions and/or components described herein.

In general, the method further includes laminating the oral care layer with the substrate and the release liner, such that the oral care layer is disposed between the substrate and the release liner, thereby preparing the oral care device. In some embodiments, the substrate is a two-layer composite including the structural layer and the wax layer disposed adjacent the structural layer. In other embodiments, the substrate is a three-layer composite including the wax layer disposed between the backing layer and the structural layer. Regardless of the particular two or three-layer configuration, the substrate may be laminated with the oral care layer in a single or multi-step process (e.g. layer by layer). Moreover, the substrate may be laminated with the oral care layer in the inactive or active form (e.g. prior to or after coating the coating the inactive oral care layer with the whitening composition). However, it will be appreciated that the oral care layer will comprise the whitening composition prior to lamination with both the release liner and the substrate.

In certain embodiments, preparing the oral care device includes laminating the oral care layer, substrate, and release liner via rollstock lamination. In such embodiments, the oral care layer, substrate, and release liner are laminated to give a rollstock laminate, and the method further includes preparing the oral care device from the rollstock laminate. Notably, the composition of the oral care layer and selection of the release liner, in view of the description above, provide for such a rollstock laminate to comprise a relatively long-lasting stability/shelf-life in terms of flexibility, potency of the whitening composition, etc. as compared to compositions outside the scope of this disclosure, e.g. those using a different adhesive agent, etc., which do not allow for formation of rollstock laminates with the structure, characteristics, and/or properties of those illustrated by the embodiments described herein.

As will be understood in view of the description above, the method typically comprises preparing an oral care laminate (e.g. the rollstock laminate) from the oral care layer. While the oral care laminate thus provided may itself be an end product (e.g. to be prepared, stored, sold, etc.), the method typically includes preparing the oral care device from the oral care laminate. The particular technique(s) and/or process(es) utilized in preparing the oral care device from the oral care laminate are not particularly limited, and may comprise any technique(s) known by those of skill in the art. Typically, preparing the oral care device from the oral care laminate (e.g. the rollstock laminate) comprises cutting (e.g. kiss cutting, die cutting, etc.) the oral care laminate into a blank of appropriate size and shape suitable for end-use or further processing.

For example, in some embodiments, the method comprises cutting the rollstock laminate into a blank having a shape typically associated with a single-use dental strip, such that the blank so prepared is the final oral care device in a form of an oral care strip. In such embodiments, the oral care strip is typically dimensioned to sufficiently cover a portion of some or all teeth in a dental arch (i.e., once installed/during use). More specifically, the oral care strip is typically dimensioned by selecting a length sufficient to cover most of the facial (e.g. buccal/labial) surfaces of teeth in the dental arch (e.g. anterior, optionally posterior teeth), and a width such that the strip may extend over the crown (e.g. incisal/occlusal surfaces) and also cover a portion of the lingual surfaces of the teeth.

In certain embodiments, the oral care strip comprises a length of from 3 to 10 cm, and a width of from 0.5 to 4 cm. However, various subranges may also be utilized, such as when the oral care strip is dimensioned for an upper or lower dental arch in particular. For example, in some embodiments, the oral care strip comprises a length of from 6 to 10 cm, such as from 7 to 9 cm, and a width of from 0.5 to 4 cm, such as from 0.8 to 2.5 cm. In other embodiments, the oral care strip comprises a length of from 3 to 7 cm, such as from 4 to 6 cm, and a width of from 1 to 2 cm. However, it is to be understood that dimensions outside these ranges may also be selected, as the oral care device may be prepared to fit a range of similarly-sized dental arches or conformed to fit a dental arch of a particular person. For example, the oral care strip device, e.g. oral care strip, may be prepared for particular use in children or smaller adults, and thus may be dimensioned proportionally smaller than the ranges described above, which are generally suitable for an adult person of average size.

In certain embodiments, the method comprises cutting the rollstock laminate into the blank, and subsequently converting the blank into an oral care tray. In such embodiments, the blank is typically dimensioned and/or shaped to include cutouts, bevels, notches, etc., such that the blank may be folded during conversion into the tray without excess material needing to be later removed. In general, the oral care tray may comprise similar dimensions as those described above with respect to the oral care strip. However, it is to be appreciated that such dimensions may apply to the oral care tray itself, i.e., rather than the blank from which it is prepared. As such, when preparing the oral care device as the tray, the blank may be over- or under-dimensioned, e.g. to account for compression, melting together, stretching, etc. during conversion.

The oral care device may comprise any shape which allows sufficient coverage of teeth, as described above, regardless of form (e.g. tray, strip, etc.). More specifically, it is to be understood that the shape/dimensions of the oral care device may be the same as those of the blank prepared from the oral care laminate (e.g. when in the strip form), or different from those properties of the blank (e.g. when in the tray form). However, during use the strip is typically disposed around a person's teeth such that the overall shape of the strip generally resembles the shape of a tray. Likewise, the tray is prepared via conversion of the blank, which generally resembles the shape of a strip (i.e., prior to use). As such, the commonalities of the shapes, dimensions, etc. between the strip, blank, and tray will be understood by those of skill in the art. For example, the tray may be similarly dimensioned to the strip when taken in plan view.

In general, the device may be substantially straight along its length, or may instead be bent (e.g. complementary to the catenary curve of a human dental arch). In some embodiments, the oral care device comprises a substantially rectangular shape. In particular embodiments, however, such as where the oral care device is particularly configured to fit over all the teeth in a person's dental arch and comprises a bend along its length, the oral care device comprises a generally horseshoe-like shape. In certain embodiments, the oral care device comprises a generally trapezoidal-like shape, i.e., having a front edge, a back edge opposite, parallel to, and shorter in length than the front edge, and opposing non-parallel side edges extending between the front and back edges. However, the oral care device may be curved, such that the front and back edges are not parallel straight-sides, but are instead complementarily curved in the same direction. As will be appreciated by those of skill in the art, such a trapezoidal-like shape may be selected when the oral care device is formed as the strip to reduce bunching when in use, and thus increase the efficiency of the device in contacting the surface of the teeth about which it is disposed. Regardless of the general/overall shape selected, the oral care device may comprise any number of cutouts, bevels, notches, chamfers, recesses, indentations, holes, etc., such that the oral care device comprises portions with individually selected shapes.

With reference to the specific embodiments described herein and illustrated in Figures, wherein like numerals generally indicate like parts throughout the several views, the oral care device is shown generally at 10.

More specifically, a planar view of the oral care device 10 in the form of the oral care strip 12 is shown in FIG. 1. As described above, the oral care strip 12 generally comprises an overall length 14 and an overall width 16, and presents a trapezoidal-like exterior shape bounded laterally by front edge 18 and back edge 20 and longitudinally by side edges 22. The side edges 22 are not necessarily perpendicular to the longitudinal axis of the oral care strip 12, but may instead be offset therefrom (e.g. as shown) and extend inwardly from the front edge 18 to the back edge 20, as represented by the relative angle 24 between the side edges 22. In this embodiment, the length 14 is from 7 to 9, alternatively from 7.5 to 8.5, alternatively from 7.5 to 8 cm. The overall width 16 is from 1.5 to 3.5, alternatively from 2 to 3, alternatively from 2.25 to 2.75 cm. The oral care strip 12 of this embodiment comprises a curve along its length, with a subwidth 26 at the apex/toe of the curve differing from the overall width 16. The subwidth 26 is from alternatively from 1 to 3, alternatively from 1.5 to 2.5, alternatively from 1.75 to 2.25 cm. The oral care strip 12 comprises indentation/cutouts 28 at the back edge 20, which allows the oral care strip 12 to be bent over a dental arch without excess bunching of material.

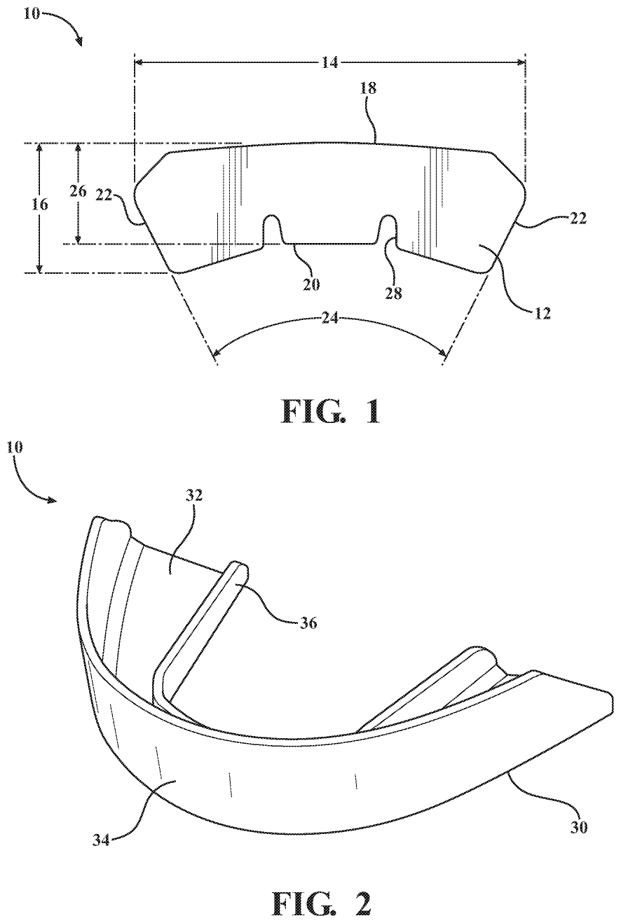

An isometric view of the oral care device 10 in the form of the oral care tray 30 is shown in FIG. 2. In particular, the oral care tray 30 of FIG. 2 is generally configured to fit an upper (i.e., superior, maxillary, etc.) dental arch of a person. The oral care tray 30 comprises a base 32, which comprises a bend along its length such that the oral care tray 30 presents a horseshoe-like shape bounded transversely by a front edge 34 and a back edge 36.

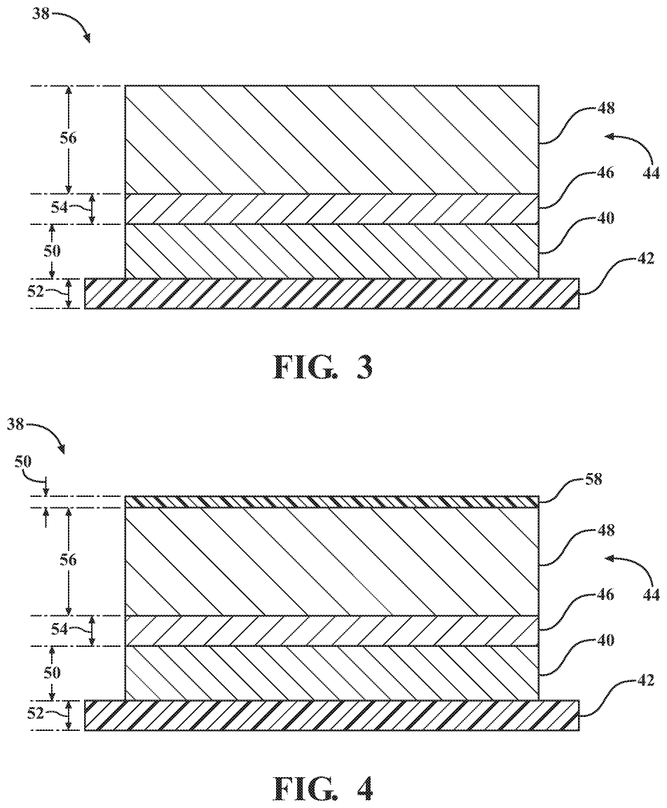

The oral care laminate is shown generally at 38 in FIGS. 3 and 4. As described above, the oral care device 10 may be prepared from the oral care laminate 38 (e.g. in the form of the strip 12 or tray 30). In certain embodiments, the oral care tray 30, as shown in FIG. 2 and described above, comprises the oral care laminate 38 shown in FIG. 3. In other embodiments, the oral care strip 12, as shown in FIG. 1 and described above, comprises the oral care laminate 38 shown in FIG. 4.

As shown in FIG. 3, one embodiment of the oral care laminate 38 includes the oral care layer 40, the release liner 42, and the substrate 44. The substrate 44 includes two layers, namely, the structural layer 46 and the wax layer 48. More specifically, the oral care laminate 38 comprises the oral care layer 40 disposed between the release liner 42 and the structural layer 46, and the wax layer 48 disposed adjacent the structural layer 46 opposite the oral care layer 40 (i.e., such that the structural layer is sandwiched between the wax layer 48 and the oral care layer 40). In this embodiment, the oral care layer 40 typically comprises an average thickness 50 of from 10 to 20 mil, the release liner 42 comprises an average thickness 52 of from 1 to 3 mil (e.g. 1.5 mil), the structural layer 46 comprises an thickness 54 corresponding to a structural material having a weight of from 22 to 28 gsm, and the wax layer 48 comprises an average thickness 56 of from 16 to 18 mil.

As shown in FIG. 4, another embodiment of the oral care laminate 38 comprises the oral care layer 40 and the substrate 44. In this embodiment, the substrate 44 includes three layers, namely, the backing layer 58 in addition to the structural layer 46 and the wax layer 48. In this embodiment, the oral care layer 40 is disposed between the release liner 42 and the structural layer 46, the wax layer 48 is disposed adjacent the structural layer 46 opposite the oral care layer 40, and the backing layer 58 is disposed adjacent the wax layer 48 opposite the structural layer 46 (i.e., such that the wax layer 48 is sandwiched between the backing layer 58 and the structural layer 46). In this embodiment, the oral care layer 40 typically comprises an average thickness 50 of from 5 to 10 mil, the release liner 42 comprises an average thickness 52 of from 2 to 4 mil (e.g. 3 mil), the structural layer 46 comprises an average thickness 54 corresponding to a structural material having a weight of from 22 to 28 gsm, the wax layer 48 comprises an average thickness 56 of from 11 to 13 mil, and the backing layer 58 comprises an average thickness 60 of from 0.2 to 0.6 mil (e.g. 0.4 mil).

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The following examples are intended to illustrate the invention and are not to be viewed in any way as limiting to the scope of the invention.

Materials/Equipment Utilized

In the examples below, a twin screw extruder (Leistritz Micro 27 40D twin screw extruder with multiple temperature zone control; screenpack: 60, 100, 200, 40 mesh; chill roller temperature: ˜18° C.) equipped with a mixing block is utilized with variable melt pump speed. The zone temperatures of the extruder observed during the examples is exemplified in Table 1 below.

TABLE 1
Twin Screw Extruder Zone Temperatures

| Function | Zone | Temperature (C.) |
|---|---|---|
| | 1 | 100 |
| INJECT | 2 | 135 |
| | 3 | 155 |
| VENT OPEN | 4 | 195 |
| | 5 | 195 |
| | 6 | 195 |
| VENT CLOSED | 7 | 195 |
| | 8 | 195 |
| | 9 | 165 |
| | 10 | 165 |
| SCREENPACK | 11 | 160 |
| PUMP | 12 | 160 |
| | 13 | 160 |
| DIE | 14 | 160 |

### Example 1: General Preparation Procedure of an Inactive Oral Care Layer

An Adhesive Agent (A), a Miscibility Component (B), and a Plasticizer (C) are combined in the twin screw extruder (melt pump speed: 15-16 RPM) to give an oral care composition, which is extruded to give an inactive oral care layer.

### Examples 2-11: Inactive Oral Care Layers

Various oral care composition are formulated and utilized in the preparation of inactive oral care layers, according to the general procedure set forth in Example 1. The components of the various oral care compositions and the parameters utilized to form the inactive oral care layers therefrom are set forth in Table 2 below. During extrusion, temperature readouts of from 166-170° C. and melt pressures of from 140-600 psi are observed.

TABLE 2
Components and Parameters of Examples 2-12

| | Adhesive | Miscibility | Plasti- |
|---|---|---|---|
| | Agent (A) | Component (B) | cizer (C) |
| Example | (wt. %) | (wt. %) | (wt. %) |
| 2 | 58.82 | 11.77 | 29.41 |
| 3 | 58.82 | 11.77 | 29.41 |
| 4 | 61.35 | 11.04 | 27.61 |
| 5 | 59.70 | 13.43 | 26.87 |
| 6 | 62.50 | 9.37 | 28.13 |
| 7 | 57.39 | 10.33 | 32.28 |
| 8 | 64.31 | 11.57 | 24.12 |
| 9 | 57.14 | 14.29 | 28.57 |
| 10 | 60.00 | 10.00 | 30.00 |
| 11 | 60.44 | 12.09 | 27.47 |
| 12 | 61.92 | 11.76 | 26.32 |

Adhesive Agent (A) is a butylated polyvinylpyrrolidone polymer having a molecular weight of around 38,000.

Miscibility Component (B) is a 1:1 methacrylic acid-ethyl acrylate copolymer.

Plasticizer (C) is a polyethylene glycol having an average molecular weight of 400 g/mol.

The inactive oral care layers are manually and visually inspected, the results of which are shown in Table 3 below.

TABLE 3
Properties of the Inactive Oral Care Lavers of Examples 2-12

| Example | Suitable for wind up | Properties (observed) |
|---|---|---|
| 2 | Yes | Golden color |
| 3 | Yes | Golden color |
| 4 | Yes | Light golden color |
| 5 | Yes | Rips/shatters under forceful manipula- |
| | | tion |
| 6 | Yes | Light yellow color |
| 7 | Yes | Clear film; spreads during wind up |
| | | (overstretch) |
| 8 | Yes | Spotted yellow color |
| 9 | Yes | N/A |
| 10 | Yes | N/A |
| 11 | Yes | Light-colored; smooth; wrinkles upon |
| | | crumple |
| 12 | Yes | Clear, flexible |

### Examples 13-18 Preparation of Active Oral Care Layers

Three liquid whitening compositions are prepared with different concentrations of whitening agent, as set forth in Table 4 below.

TABLE 4
Liquid Whitening Compositions (LWCs) 1-3

| Component | LWC-1 | LWC-2 | LWC-3 |
|---|---|---|---|
| Carrier 1 (wt. %) | 77.9000 | 64.2787 | 53.0890 |
| Whitening Agent 1 (wt. %) | 20.0000 | 33.8380 | 45.0000 |
| Flavorant 1 (wt. %) | 1.2500 | 1.0000 | 1.0000 |
| Chelator 1 (wt. %) | 0.5000 | 0.5000 | 0.5000 |
| Surfactant 1 (wt. %) | 0.3000 | 0.3000 | 0.3000 |
| pH Modifier 1 (wt. %) | 0.0500 | 0.0833 | 0.1110 |

Carrier 1 is water.

Whitening Agent 1 is hydrogen peroxide.

Flavorant 1 is sodium saccharin.

Chelator 1 is disodium EDTA.

Surfactant 1 is Poloxamer 407.

pH Modifier 1 is sodium hydroxide.

Various active oral care layers are prepared by coating an inactive oral care layer with a liquid whitening composition, as set forth in Table 5 below.

TABLE 5
Active Oral Care Layer Preparation

| Example | Inactive OCL | Liquid Whitening Composition |
|---|---|---|
| 13 | IOCL-1 | LWC-1 |
| 14 | IOCL-1 | LWC-2 |
| 15 | IOCL-1 | LWC-3 |
| 16 | IOCL-2 | LWC-1 |
| 17 | IOCL-2 | LWC-2 |
| 18 | IOCL-2 | LWC-3 |

IOCL-1 is the inactive oral care layer prepared in Example 2 above.

IOCL-2 is the inactive oral care layer prepared in Example 3 above.

LWC-1 is the liquid whitening composition prepared in Table 3 above.

LWC-2 is the liquid whitening composition prepared in Table 3 above.

LWC-3 is the liquid whitening composition prepared in Table 3 above.

The final formulations of the active oral care layers of Examples 13-18 are set forth in Table 6 below.

TABLE 6
Active Oral Care Layers of Examples 13-18

| Component | Ex. 13 | Ex. 14 | Ex. 15 | Ex. 16 | Ex. 17 | Ex. 18 |
|---|---|---|---|---|---|---|
| Adhesive Agent (A) (wt. %) | 44.1159 | 41.4390 | 41.4390 | 44.1159 | 41.4390 | 41.4390 |
| Miscibility Component (B) (wt. %) | 8.8232 | 8.2878 | 8.2878 | 8.8232 | 8.2878 | 8.2878 |
| Plasticizer (C) (wt. %) | 22.058 | 20.7195 | 20.7195 | 22.058 | 20.7195 | 20.7195 |
| Carrier 1 (wt. %) | 19.4773 | 18.9967 | 17.6964 | 19.4773 | 18.996 | 17.696 |
| Whitening Agent 1 (wt. %) | 5.0006 | 10.0004 | 15.0000 | 5.0006 | 10.0004 | 15.0000 |
| Flavorant 1 (wt. %) | 0.3125 | 0.2955 | 0.3333 | 0.3125 | 0.2955 | 0.3333 |
| Chelator 1 (wt. %) | 0.1250 | 0.1478 | 0.1667 | 0.1250 | 0.1478 | 0.1667 |
| Surfactant 1 (wt. %) | 0.0750 | 0.0887 | 0.1000 | 0.0750 | 0.0887 | 0.1000 |
| pH Modifier 1 (wt. %) | 0.0125 | 0.0246 | 0.0370 | 0.0125 | 0.0246 | 0.0370 |

Adhesive Agent (A) is a butylated polyvinylpyrrolidone polymer having a molecular weight of around 38,000.

Miscibility Component (B) is a 1:1 methacrylic acid-ethyl acrylate copolymer.

Plasticizer (C) is a polyethylene glycol having an average molecular weight of 400 g/mol.

Carrier 1 is water.

Whitening Agent 1 is hydrogen peroxide.

Flavorant 1 is sodium saccharin.

Chelator 1 is disodium EDTA.

Surfactant 1 is Poloxamer 407.

pH Modifier 1 is sodium hydroxide.

### Examples 19-21 Preparation of Oral Care Strips with 5 mm Active OCL

Oral care strips are prepared via rollstock laminating and converting the active oral care layers prepared in Examples 13-15 above. Each active oral care layer (5 mm) is rollstock laminated with a polyethylene backing layer (0.4 mm), a non-woven structural layer (22-28 g/m²), a wax layer (11-13 mm), and a release liner (3.0 mm), to prepare oral care laminates having a laminate structure as shown in FIG. 4.

Oral care strips are prepared from the oral care laminates. In particular, each oral care laminate is kiss-cut to prepare oral care strips having a flat shape as shown in FIG. 1 and packaged (weld seal foil pouch, four-sided) on a converting machine (Delta ModTech Rotary Converter and Packager).

### Examples 22-24 Preparation of Oral Care Trays with 10 mm Active OCL

Oral care strips are prepared via rollstock laminating and converting the active oral care layers prepared in Examples 16-18 above. Each active oral care layer (10 mm) is rollstock laminated with a non-woven structural layer (22-28 g/m²), a wax layer (16-18 mm), and a release liner (1.5 mm) to prepare oral care laminates having a laminate structure as shown in FIG. 3.

Oral care trays are prepared from the oral care laminates. In particular, each oral care laminate is cut and formed into an oral care tray having a tray shape as shown in FIG. 2 and packaged (PVC blister packaging with a peelable foil lidstock) on a sequential converting and packaging machine assembly with automated web handling, cutting, forming, robotic pick and place, inspection, and packaging operations.

### Comparative Examples 1-19

Various compositions are formulated and extruded to prepare films according to the general procedures set forth in Examples 1, utilizing the components and parameters set forth in Table 7 below.

TABLE 7
Components and Parameters of Comparative Examples 1-19

| | Adhesive | Miscibility | Plasti- | |
|---|---|---|---|---|
| Comparative | Agent (A-2) | Component (B) | cizer (C) | Additive 1 |
| Example | (wt. %) | (wt. %) | (wt. %) | (wt. %) |
| 1 | 58 | 12 | 30 | 0 |
| 2 | 68 | 12 | 20 | 0 |
| 3 | 74 | 6 | 20 | 0 |
| 4 | 71 | 6 | 23 | 0 |
| 5 | 68 | 6 | 26 | 0 |
| 6 | 66 | 8 | 26 | 0 |
| 7 | 62 | 10 | 28 | 0 |
| 8 | 64 | 12 | 22 | 2 |
| 9 | 66 | 0 | 34 | 0 |
| 10 | 75 | 0 | 25 | 0 |
| 11 | 70 | 0 | 30 | 0 |
| 12 | 72.5 | 0 | 27.5 | 0 |
| 13 | 58 | 12 | 30 | 0 |
| 14 | 54.5 | 13 | 32.5 | 0 |
| 15 | 51 | 14 | 35 | 0 |
| 16 | 47.5 | 15 | 37.5 | 0 |
| 17 | 44 | 16 | 40 | 0 |
| 18 | 40.5 | 17 | 42.5 | 0 |
| 19 | 37 | 18 | 45 | 0 |

Adhesive Agent (A-2) is a vinylpyrrolidone-vinyl acetate- (VP/VA) copolymer having a 1-vinyl-2-pyrrolidone to vinyl acetate ratio of 6:4, by mass, and a weight-average molecular weight of from 45,000 to 70,000.

Miscibility Component (B) is a 1:1 methacrylic acid-ethyl acrylate copolymer.

Plasticizer (C) is a polyethylene glycol having an average molecular weight of 400 g/mol.

Additive 1 is sodium carboxymethyl cellulose.

In each of Comparative Examples 1-12, the extruded sheets are brittle and shatter easily upon manipulation. The extruded sheets of Comparative Examples 13-19 are flexible with no observable cracking/breaking/shattering on cure.

Each of the extruded sheets is then coated with water and/or aqueous hydrogen peroxide and laminated according to the procedures set forth above. The extruded sheets of Comparative Examples 1-19 exhibit poor cohesion with water and liquid whitening compositions, loss of structural/mechanical integrity upon coating with water and/or the liquid whitening composition, and, when lamination is successful, delamination upon removal of the release liner.

### Example 25 and Comparative Examples 20-21: Solvent Casting

Various compositions are formulated utilizing the components and parameters set forth in Table 8 below. In particular, in each of components (A)-(C) is combined into a solvent (ethanol) and mixed (FlackTek DAC 150FVZ-K SpeedMixer) to give a homogeneous mixture, which is then cast onto a polyethylene terephthalate (PET) layer and dried (ambient conditions) to give a film. Each film is then coated with aqueous hydrogen peroxide and laminated according to the procedures set forth above.

TABLE 8
Components and Parameters of Example
25 and Comparative Examples 20-21

| | | Comp. | Comp. |
|---|---|---|---|
| Component | Ex. 25 | Ex. 20 | Ex. 21 |
| Adhesive Agent (A-1) (wt. %) | 58.82 | — | — |
| Adhesive Agent (A-3) (wt. %) | — | 58.82 | — |
| Adhesive Agent (A-4) (wt. %) | — | — | 58.82 |
| Miscibility Component (B) (wt. %) | 11.76 | 11.76 | 11.76 |
| Plasticizer (C) (wt. %) | 29.41 | 29.41 | 29.41 |

Adhesive Agent (A-1) is a butylated polyvinylpyrrolidone polymer having a molecular weight of around 38,000.

Adhesive Agent (A-3) is a copolymer of N-vinyl-2-pyrrolidone, acrylic acid, and lauryl methacrylate (i.e., VP/acrylates/lauryl methacrylate copolymer).

Adhesive Agent (A-4) is a 60:40 linear random copolymer of N-vinyl-2-pyrrolidone and vinyl acetate (i.e., 6:4 VP/VA copolymer).

Miscibility Component (B) is a 1:1 methacrylic acid-ethyl acrylate copolymer.

Plasticizer (C) is a polyethylene glycol having an average molecular weight of 400 g/mol.

The cast film of Example 25 was pliable and laminated successfully with hydrogen peroxide mix. The cast film of Comparative Example 20 was hard and brittle, and unsuitable for use in lamination. The cast film of Comparative Example 21 was flexible but lost integrity and cohesion when laminated with the aqueous peroxide.

The invention has been described in an illustrative manner, and it is to be understood that the terminology which has been used is intended to be in the nature of words of description rather than of limitation. Obviously, many modifications and variations of the present invention are possible in light of the above teachings. The invention may be practiced otherwise than as specifically described.

1. An oral care composition, comprising:
(A) an adhesive agent comprising an alkylated polyvinylpyrrolidone;
(B) a miscibility component comprising an acrylic acid-acrylate copolymer; and
(C) a plasticizer comprising a polyalkylene glycol.

2. The oral care composition of claim 1, wherein: (i) the alkylated polyvinylpyrrolidone is a butylated polyvinylpyrrolidone; (ii) the alkylated polyvinylpyrrolidone comprises an average molecular weight of from 30,000 to 50,000; (iii) the adhesive agent (A) is present in the oral care composition in an amount of from 40 to 60 wt. % based on the total weight of the oral care composition; or (iv) any combination of (i)-(iii).

3. The oral care composition of claim 1, wherein: (i) the acrylic acid-acrylate copolymer comprises an acid-ester ratio of from 1:1 to 1:2; (ii) the acrylic acid-acrylate copolymer is a methacrylic acid-ethyl acrylate copolymer; (iii) the miscibility component (B) is present in the oral care composition in an amount of from 8 to 12 wt. % based on the total weight of the oral care composition; or (iv) any combination of (i)-(iii).

4. The oral care composition of claim 1, wherein: (i) the polyalkylene glycol comprises an average molecular weight of from 300 to 500; (ii) the polyalkylene glycol is a polyethylene glycol; (iii) the plasticizer (C) is present in the oral care composition in an amount of from 20 to 30 wt. % based on the total weight of the oral care composition; or (iv) any combination of (i)-(iii).

5. The oral care composition of claim 1, further comprising a whitening agent.

6. The oral care composition of claim 5, wherein: (i) the whitening agent comprises hydrogen peroxide, and the hydrogen peroxide is present in the oral care composition in an amount of from 5 to 15 wt. % based on the total weight of the oral care composition; (ii) the whitening agent comprises a carbamide peroxide, and the carbamide peroxide is present in the oral care composition in an amount of from 15 to 45 wt. % based on the total weight of the oral care composition; (iii) the whitening agent comprises a phthalimidoperoxycaproic acid, and the phthalimidoperoxycaproic acid is present in the oral care composition in an amount of from 0.1 to 15 wt. % based on the total weight of the oral care composition; or (iv) any combination of (i)-(iii).

7. The oral care composition of claim 1, further comprising: (i) a flavoring agent; (ii) a surfactant; (iii) a thickener; (iv) a pH modifier; (v) a buffer; (vi) a filler; (vii) a preservative; (viii) a coloring agent; (ix) a viscosity modifier; or (x) any combination of (i)-(ix).

8. An oral care device, comprising:
an oral care layer comprising an oral care composition and a whitening agent;
wherein the oral care composition is the oral care composition of claim 1.

9. The oral care device of claim 8, wherein the oral care layer is further defined as: (i) an extruded film; or (ii) a solvent-cast film.

10. The oral care device of claim 8, wherein the oral care layer comprises an average thickness of from 5 to 10 mil.

11. The oral care device of claim 8, wherein the oral care layer comprises an average thickness of from 10 to 20 mil.

12. The oral care device of claim 8, comprising a laminate of the oral care layer and: (i) a substrate disposed adjacent the oral care layer; (ii) a release liner disposed adjacent the oral care layer; or (iii) both (i) and (ii).

13. The oral care device of claim 12, wherein the laminate comprises the substrate and wherein the substrate comprises: (i) a structural layer; (ii) a wax layer; (iii) a backing layer; or (iv) any combination of (i)-(iii).

14. The oral device of claim 13, wherein the substrate comprises the wax layer and the structural layer, and wherein the structural layer is disposed between the wax layer and the oral care layer.

15. The oral device of claim 14, wherein the substrate further comprises the backing layer, and wherein the backing layer is disposed adjacent the wax layer opposite the structural layer.

16. The oral care device of claim 13, wherein the laminate further comprises the release liner, and wherein the oral care layer is disposed between the release liner and the substrate.

17. A method of preparing an oral care device, said method comprising:
extruding an oral care composition to give an oral care layer, the oral care composition comprising (A) an adhesive agent comprising an alkylated polyvinylpyrrolidone, (B) a miscibility component comprising an acrylic acid-acrylate copolymer, and (C) a plasticizer comprising a polyalkylene glycol; and
laminating the oral care layer with a substrate and a release liner such that the oral care layer is disposed between the substrate and the release liner, thereby preparing the oral care device.

18. The method of claim 17, wherein extruding the oral care composition prepares an inactive oral care layer, and wherein the method further comprises disposing a whitening composition comprising a whitening agent onto the inactive oral care layer to give the oral care layer.

19. The method of claim 17, wherein laminating the oral care layer, the substrate, and the release liner gives a rollstock laminate, and wherein the method further comprises forming the oral care device from the rollstock laminate.

20. The method of claim 19, wherein forming the oral care device from the rollstock laminate comprises cutting the rollstock laminate into an oral care strip, and optionally converting the oral care strip into an oral care tray.
